SEQUENCE LISTING (15 sequences)

SEQ ID NO: 1 - protein, 11 aa, synthetic construct
YARAAARQARA

SEQ ID NO: 2 - protein, 21 aa, synthetic construct
WLRRIKAWLRRIKAWLRRIKA

SEQ ID NO: 3 - protein, 11 aa, synthetic construct
YGRKKRRQRRR

SEQ ID NO: 4 - protein, 16 aa, synthetic construct
RQIKIYFQNRRMKWKK

SEQ ID NO: 5 - protein, 34 aa, synthetic construct
DAATATRGRSAASRPTERPRAPARSASRPRRPVE

SEQ ID NO: 6 - protein, 16 aa, synthetic construct
AAVALLPAVLLALLAP

SEQ ID NO: 7 - protein, 26 aa, synthetic construct
GWTLNSAGYLLKINLKALAALAKKIL

SEQ ID NO: 8 - protein, 18 aa, synthetic construct
KLALKLALKALKAALKLA

SEQ ID NO: 9 - protein, 15 aa, synthetic construct
RRRRNRTRRNRRRVR

SEQ ID NO: 10 - protein, 21 aa, synthetic construct
KETWWETWWTEWSQPKKKRKV

SEQ ID NO: 11 - DNA, 17 bp, synthetic construct
  source 1..17: mol_type = other DNA
gaaaagggtcgcggggg

SEQ ID NO: 12 - DNA, 20 bp, synthetic construct
  source 1..20: mol_type = other DNA
atgttcagagaggtccccag

SEQ ID NO: 13 - DNA, 17 bp, synthetic construct
  source 1..17: mol_type = other DNA
cccccgcgacccttttc

SEQ ID NO: 14 - DNA, 20 bp, synthetic construct
  source 1..20: mol_type = other DNA
aggtccagaaagatgccaag

SEQ ID NO: 15 - protein, 11 aa, synthetic construct
RRRRRRRRRRR

(19) United States
(12) Patent Application Publication
(10) Pub. No.: US 2024/0156856 A1
(43) Pub. Date: May 16, 2024

(54) NOVEL APPLICATIONS OF HYALURONIC ACID FOR TREATMENT OF PAIN AND PRURITIS

(71) Applicant: THE REGENTS OF THE UNIVERSITY OF CALIFORNIA, Oakland, CA (US)

(72) Inventor: JON LEVINE, Oakland, CA (US)

(21) Appl. No.: 18/323,122

(22) Filed: May 24, 2023

Related U.S. Application Data
(60) Provisional application No. 63/375,769, filed on Sep. 15, 2022.
(60) Provisional application No. 63/345,353, filed on May 24, 2022.

(51) Int. Cl. A61K 31/728; A61K 9/00; A61K 47/20; A61P 29/00

(52) U.S. Cl. CPC A61K 31/728; A61K 9/0014; A61K 47/20; A61P 29/00

(57) ABSTRACT

The disclosure is generally related to therapeutic compositions and methods for use in the treatment of somatosensory conditions, including pain and pruritis. In some aspects, the compositions comprise hyaluronic acid having a therapeutically effective molecular weight and one or more transdermal delivery agents.

### CROSS-REFERENCE TO RELATED APPLICATIONS

This application claims the priority benefit under 35 U.S.C. § 119(e) of U.S. Provisional Application No. 63/345,353, filed May 24, 2022 and U.S. Provisional Application No. 63/375,769, filed Sep. 15, 2022, which are incorporated herein by reference in their entirety.

### STATEMENT REGARDING FEDERALLY SPONSORED RESEARCH OR DEVELOPMENT

This invention was made with government support under grants R01 AR075334 and R01 CA250017 awarded by The National Institutes of Health. The government has certain rights in the invention.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “50022R_SeqListing.xml”, which was created on May 20, 2023 and is 2,048,580 bytes in size. The subject matter of the Sequence Listing is incorporated herein in its entirety by reference.

### BACKGROUND

Sensory neurons in the skin and other parts of the body transduce mechanical, chemical and other stimuli to guide animal behavior. One important subset of sensory neurons are nociceptors activity in which mediate pain. Another subset of sensory neurons called pruriceptors, mediate the sensation of pruritis or itch. Both pain and itch can be pathological in certain contexts. In the case of pain, neuropathic pain for example encompasses a suite of painful conditions suffered by many millions of subjects worldwide. Likewise, for a large pool of persons, pathological pruritus causes significant reduction in quality of life due to discomfort and associated sleep disruption.

Meanwhile, hyaluronic acid (HA), a major constituent of the extracellular matrix, is a bioactive molecule implicated in numerous biological processes and systems in the body. In the biological arts, general reference is made to “low molecular weight” and “high molecular weight” hyaluronic acid polymers, and various biological effects are ascribed thereto. Opposing biological effects of “low” and “high” molecular weight hyaluronic acid have been observed in various contexts, for example pro-inflammatory effects by lower molecular weight hyaluronic acid polymers and anti-inflammatory effects for higher molecular weight hyaluronic acid. However, despite the biological importance of distinguishing these two classes of hyaluronic acid, there is a general inconsistency in the art as to what constitutes “low” molecular weight hyaluronan and what constitutes “high” molecular weight hyaluronan. For example, as noted in Cyphert et al., 2015, Size Matters: Molecular Weight Specificity of Hyaluronan Effects in Cell Biology. International Journal of Cell Biology, Volume 2015, Article ID 563818:
“An additional impediment on the elucidation of size-dependent HA signaling and biological effects is the confusing language that is used in scientific publications. While everyone seems to agree on designating HA over 1 million Da “high molecular weight,” the nomenclature of smaller-size HA is nebulous.”
   Accordingly, with respect to biological or therapeutic effects of hyaluronic acid on the treatment of somatosensory disorders, there is a need in the art for increased specificity in reference to hyaluronan size such that the skilled artisan may determine with accuracy what size polymers will impart desired biological effects.

In the case of neuropathic pain, it has been shown that hyperalgesia can be attenuated by intradermal administration of high molecular weight hyaluronic acid, also called hyaluronan. For example, as described in Bonet et al., 2020, Mechanisms Mediating High-Molecular-Weight Hyaluronan-Induced Antihyperalgesia, Journal of Neuroscience, 40:6477-6488, signaling pathways by which high molecular weight hyaluronan induce anti-hyperalgesia were investigated, elucidating a central role for CD44, a receptor in pain sensory neurons (nociceptors), and its downstream effectors in inhibiting pain. Therein it was demonstrated that intradermal injection of hyaluronic acid polymers having a molecular weight range of 500-1200 kDa had substantial anti-hyperalgesia effects. These prior art efforts have shed much light on the signaling pathways by which hyaluronic acid can act to mitigate neuropathic pain. However, there remains a need in the art for an increased understanding of how the molecular weight of hyaluronic acid mediates its action on nociceptors, for example, to reconcile the paradoxical effects of low and high molecular weight polymers on pain perception. Such an understanding would enable the practical application of hyaluronan-based treatments to address neuropathic and other types of pain.

Additionally, there remains a need in the art for improved therapeutic approaches to pain and pruritis by the application of hyaluronan. As above, previous research efforts have demonstrated that intradermal application of hyaluronan can have local anti-hyperalgesia effects. However, practical application of this discovery in the clinic has not been achieved. One obstacle is that intradermal injection is painful, and requires administration by trained health care professionals, resulting in more costly and less convenient treatment, and is only effective at the site of injection. Accordingly, there remains a need in the art for more convenient and effective forms of hyaluronan administration to treat neuropathic pain.

Meanwhile, in the context of pruritis, treatment of itch is known in the art to be challenging. Current therapeutic interventions for pruritus, such as administration of antihistamines, are non-specific, often ineffective for many subjects and produce side effects. Thus, there remains a strong need in the art for novel therapeutics for treating all the various forms of pruritis.

### SUMMARY

Disclosed herein are various novel therapeutic compositions and methods for use in the treatment of somatosensory conditions, including pain and pruritis.

In a first aspect, the scope of the invention encompasses a novel method of treating pain, for example, neuropathic pain, by the topical administration of high molecular weight hyaluronic acid. This invention is based on the novel and unprecedented demonstration of topical hyaluronic acid application to achieve anti-hyperalgesia. This discovery provides the art with a novel tool for the treatment of pain by conveniently administered and inexpensive topical formulations of hyaluronic acid.

In another aspect, the scope of the invention encompasses a novel method of treating pain, for example, neuropathic pain, by the administration of hyaluronic acid polymers of a selected, therapeutically effective molecular weight which has anti-hyperalgesia effects. As outlined above, differently-sized hyaluronic acid polymers can have opposing effects on nociceptors and pain perception. By extensive experimentation, the inventor of the present disclosure has elucidated a molecular weight breakpoint, above which hyaluronic acid polymers have anti-hyperalgesia properties. This application provides the art with the means to select hyaluronic acid polymers of therapeutically effective size, and for the development of effective formulations, particularly topical formulations.

In another aspect, the scope of the invention encompasses a novel topical formulation for the treatment of pain, for example, neuropathic pain, comprising hyaluronic acid polymers in combination with the carrier DMSO. The inventor of the present disclosure has devised formulations of DMSO and hyaluronan that are particularly effective in activating anti-hyperalgesia signaling to treat pain.

In another aspect, the scope of the invention encompasses the novel treatment of pruritic conditions by the administration of hyaluronic acid, for example, topical administration. The inventor of the present disclosure has surprisingly determined that pruritic conditions can be treated by administration of hyaluronic acid. This is an unexpected result as pain inhibits pruritis, so decreasing pain would be expected to increase pruritis. This discovery provides the art with novel tools for treating various forms of pruritus.

The foregoing inventions are described in detail next.

### BRIEF DESCRIPTION OF THE DRAWINGS

FIG. 1 shows that topical administration of 500-1200 kDa hyaluronan (HA) in DMSO vehicle inhibits PGE₂ hyperalgesia in male and female rats. PGE₂ (100 ng/5 μL, i.d.) was injected on the dorsum of the hind paw of male and female rats. Following the development of mechanical hyperalgesia 500-1200 kDa hyaluronan (2 μg/μL in a volume of 30 μL), or its DMSO vehicle (30 μL), was applied at the site of nociceptive threshold testing on the dorsum of the hind paw. Mechanical nociceptive threshold was evaluated before and again 30 and 60 min after PGE₂. A. Topical administration of high molecular weight hyaluronan (HMWH) 500-1200 kDa hyaluronan in DMSO vehicle attenuated PGE₂-induced hyperalgesia, in male rats (repeated measures one-way ANOVA, 30 min: Treatment F_{(1.35,6.75)}=40.11, P=0.0003, Tukey's multiple comparison test PGE₂ alone vs. PGE₂+hyaluronan P=0.0003, PGE₂+hyaluronan vs PGE₂+DMSO vehicle P=0.0014; 60 min: Treatment F_{(1.84,9.2)}=40.15, P<0.0001, Tukey's multiple comparison test PGE₂ alone vs. PGE₂+hyaluronan P=0.0009, PGE₂+hyaluronan vs PGE₂+DMSO vehicle P=0.0039). B. Topical administration of 500-1200 kDa hyaluronan in DMSO vehicle also attenuated PGE₂-induced hyperalgesia in female rats (repeated measures one-way ANOVA, 30 min: Treatment F_{(1.85,9.26)}=60.23, P<0.0001, Tukey's multiple comparison test PGE₂ alone vs. PGE₂+hyaluronan P=0.0005, PGE₂+hyaluronan vs PGE₂+vehicle P=0.0011; 60 min: Treatment F_{(1.39,6.93)}=29.87, P=0.0006, Tukey's multiple comparison test PGE₂ alone vs. PGE₂+hyaluronan P=0.004, PGE₂+hyaluronan vs PGE₂+DMSO vehicle P=0.0004). n=6 per group.

FIG. 2 shows the dose-response relationship for anti-hyperalgesia induced by topical hyaluronan in rats with CIPN. A. Male rats received oxaliplatin (2 mg/kg, i.v.) on day 0. On day 7, 500-1200 kDa hyaluronan, at three different doses (1, 2 or 4 μg/μL or DMSO vehicle, each in a volume of 30 μL) was administered topically on the dorsum of the hind paw, at the site of nociceptive threshold testing. Mechanical nociceptive threshold was evaluated before and 7 days after administration of oxaliplatin, and again 10, 30, 60 and 120 min after topical hyaluronan in DMSO vehicle. Oxaliplatin decreased mechanical nociceptive threshold (i.e., produced hyperalgesia) (paired Student's t-test, t(23)=15.62, P<0.0001). Topical administration of 500-1200 kDa hyaluronan dose-dependently attenuated the hyperalgesia induced by oxaliplatin (Two-way repeated measures ANOVA, dose F_{(3.20=10.08)}, P=0.0003). n=6 per group. B. Male rats received oxaliplatin (2 mg/kg, i.v.) on day 0. On day 7 after oxaliplatin administration, 500-1200 kDa hyaluronan (2 μg/μL in a volume of 30 μL) dissolved in 0.9% saline (without DMSO) or vehicle (0.9% saline, 30 μL) alone was administered topically on the dorsum of the hind paw, at the site of nociceptive threshold testing. Mechanical nociceptive threshold was evaluated before oxaliplatin and 7 days after its administration, and again 30, 60 and 120 min after topical administration of hyaluronan. Oxaliplatin decreased mechanical nociceptive threshold (i.e., produced hyperalgesia) (paired Student's t-test, t(11)=14.65, P<0.0001). Topical administration of 500-1200 kDa hyaluronan dissolved in saline, without DMSO, did not attenuate oxaliplatin-induced hyperalgesia (Two-way repeated measures ANOVA, dose F_{(1,10)}=0.11, P=0.75). n=6 per group.

FIG. 3 shows that topical hyaluronan-induced anti-hyperalgesia for CIPN is sexually dimorphic. Male and female rats received oxaliplatin (2 mg/kg, i.v.) on day 0. On day 7, 500-1200 kDa hyaluronan (2 μg/μL in a volume of 30 μL) or its DMSO vehicle alone (30 μL) was applied to the dorsum of the hind paw, at the site of nociceptive threshold testing. Groups of male and female rats received paclitaxel (1 mg/kg, i.p.), every other day for a total of 4 doses (days 0, 2, 4 and 6). On day 7, approximately 24 h after the last dose of paclitaxel, hyaluronan (2 μg/μL in a volume of 30 μL) or DMSO vehicle (30 μL) was applied topically on the dorsum of the hind paw, at the site of nociceptive threshold testing. Additional groups of rats received bortezomib (0.2 mg/kg, i.v., every other day for a total of 4 doses). On day 7, approximately 24 h after the last dose of bortezomib, 500-1200 kDa hyaluronan (2 μg/μL in a volume of 30 μL) or DMSO vehicle (30 μL) was applied topically on the dorsum of the hind paw, at the site of nociceptive threshold testing. Mechanical nociceptive threshold was evaluated before administration of oxaliplatin, paclitaxel or bortezomib and 7 days after, and again 30, 60 and 120 min after topical administration of hyaluronan. A. Oxaliplatin decreased mechanical nociceptive threshold (i.e., produced hyperalgesia) in male rats. Topical administration of 500-1200 kDa hyaluronan attenuated hyperalgesia induced by oxaliplatin (Two-way repeated measures ANOVA, dose F_{(1,10)}=24.98, P=0.0005) (results in this figure are reproduced from FIG. 2, panel A, for comparison). B. Paclitaxel decreased mechanical nociceptive threshold (i.e., produced hyperalgesia) in male rats. In this group of rats, topical administration of 500-1200 kDa hyaluronan attenuated the hyperalgesia induced by paclitaxel (Two-way repeated measures ANOVA, dose F_{(1,10)}=78.74, P<0.0001). C. Bortezomib decreased mechanical nociceptive threshold (i.e., produced hyperalgesia) in male rats. Topical administration of HMWH attenuated hyperalgesia induced by bortezomib (Two-way repeated measures ANOVA, dose F_{(1,10)}=39.39, P<0.0001). D. In female rats, hyperalgesia induced by oxaliplatin was not attenuated by topical administration of 500-1200 kDa hyaluronan (Two-way repeated measures ANOVA, dose F_{(1,10)}=0.039, P=0.8464). n=6 per group. E. In female rats, hyperalgesia induced by paclitaxel was not attenuated by topical administration of 500-1200 kDa hyaluronan (Two-way repeated measures ANOVA, dose F_{(1,10)}=0.004, P=0.9503). n=6 per group. F. In female rats, hyperalgesia induced by bortezomib was not attenuated by topical administration of 500-1200 kDa hyaluronan (Two-way repeated measures ANOVA, dose F_{(0,10)}=1.41, P=0.2471).

FIG. 4 shows that sex hormones attenuate hyaluronan-induced anti-hyperalgesia in female rats. A. A group of female rats underwent surgical ovariectomy 3 weeks prior to receiving oxaliplatin (2 mg/kg, i.v.), administered on day 0. On day 7 they received 500-1200 kDa hyaluronan (2 μg/μL in a volume of 30 μL) or DMSO vehicle (30 μL), administered topically on the dorsum of the hind paw, at the site of nociceptive threshold testing. Mechanical nociceptive threshold was evaluated before and 7 days after oxaliplatin, and again 30, 60 and 120 min after topical hyaluronan. Oxaliplatin decreased mechanical nociceptive threshold, measured 7 days after its administration. Topical administration of 500-1200 kDa hyaluronan attenuated the hyperalgesia induced by oxaliplatin in ovariectomized (Two-way repeated measures ANOVA, dose F_{(1,10)}=37.75, P=0.0001), in contrast to gonad intact, female rats (FIG. 3C). n=6 per group. B. Another group of female rats underwent ovariectomy 3 weeks prior to receiving a first dose of paclitaxel (1 mg/kg, i.p.), which was administered every other day for a total of 4 doses (days 0, 2, 4 and 6). Seven days after the first dose of paclitaxel, rats were treated with 500-1200 kDa hyaluronan (2 μg/μL in a volume of 30 μL) or DMSO vehicle (30 μL), applied topically on the dorsum of the hind paw, at the site of nociceptive threshold testing. Mechanical nociceptive threshold was evaluated before and 7 days after the first dose of paclitaxel, and again 30, 60 and 120 min after topical hyaluronan. Paclitaxel decreased mechanical nociceptive threshold, measured 7 days after its first dose, in ovariectomized and gonad-intact female rats. Topical 500-1200 kDa hyaluronan attenuated the hyperalgesia induced by paclitaxel in gonadectomized (Two-way repeated measures ANOVA, dose F_{(1,10)}=66.27, P<0.0001) but not gonad intact female rats (FIG. 3D). n=6 per group. C. Female rats received oxaliplatin (2 mg/kg, i.v.) on day 0. Four days later, they were treated i.t. with ODN antisense or mismatch (120 μg/20 μL, i.t.) for GPR30 mRNA, daily for 3 consecutive days. On day 7, approximately 24 h after the last dose of ODN, 500-1200 kDa hyaluronan (2 μg/μL in a volume of 30 μL) or its vehicle (30 μL) was administered topically on the dorsum of the hind paw, at the site of nociceptive threshold testing. Mechanical nociceptive threshold was evaluated before oxaliplatin, 3 and 7 days after its administration, and again 30, 60 and 120 min after topical hyaluronan. Oxaliplatin decreased mechanical nociceptive threshold in both GPR30 antisense- and mismatch-ODN treated rats. However, 500-1200 kDa hyaluronan attenuated the hyperalgesia induced by oxaliplatin only in the GPR30 antisense-treated group (Two-way repeated measures ANOVA, dose F_{(1,10)}=24.83, P=0.0006). n=6 per group. D. Another group of female rats received paclitaxel (1 mg/kg, i.p. every other day for a total of 4 doses). Starting four days after the 1st dose of paclitaxel, rats were treated with antisense or mismatch ODN for GPR30 mRNA (120 μg/20 μL, i.t.), daily for 3 consecutive days. On day 7, approximately 24 h after the last ODN dose and the last dose of paclitaxel, 500-1200 kDa hyaluronan (2 μg/μL in a volume of 30 μL) or DMSO vehicle (30 μL) was administered topically on the dorsum of the hind paw, at the site of nociceptive threshold testing. Mechanical nociceptive threshold was evaluated before paclitaxel, 3 and 7 days after the first administration of paclitaxel, and again 30, 60 and 120 min after topical 500-1200 kDa hyaluronan. Paclitaxel decreased mechanical nociceptive threshold in both GPR30 antisense- (one-way repeated measures ANOVA F_{(1.53,7.63)} P<0.0001) and mismatch-treated groups (one-way repeated measures ANOVA F_{(1.13,5.65)} P=0.0002). However, 500-1200 kDa hyaluronan only attenuated the hyperalgesia induced by paclitaxel in the GPR30 antisense-treated group (Two-way repeated measures ANOVA, dose F_{(1,10)}=15.51, P=0.0028). n=6 per group.

FIG. 5 shows that concentration of DMSO effects hyaluronan-induced anti-hyperalgesia. A. Male rats received oxaliplatin (2 mg/kg, i.v.) on day 0. On day 7, 500-1200 kDa hyaluronan (2 μg/μL in a volume of 30 μL) in six different percentages of DMSO (75%, 50%, 25%, 20%, 15% and 5% DMSO) or DMSO vehicle (30 μL) was administered topically on the dorsum of the hind paw at the site of nociceptive threshold testing. Mechanical nociceptive threshold was evaluated before oxaliplatin and 7 days after its administration, and again 30, 60 and 120 min after topical hyaluronan. Results are presented as mechanical nociceptive threshold in grams. Oxaliplatin decreased mechanical nociceptive threshold (i.e., produced hyperalgesia; paired Student's t-test, baseline versus post-oxaliplatin before hyaluronan, for each group: Vehicle P=0.003956). However, topical administration of HMWH in 75%, 50%, 25% and 20% produced anti-hyperalgesia (75% of DMSO P<0.0001, 50% of DMSO P<0.0001, 25% of DMSO P<0.0001, 20% of DMSO P<0.0001). Topical administration of HMWH in 15% and 5% of DMSO did not produce anti-hyperalgesia (15% of DMSO P>0.99, 5% of DMSO P=0.99). Two-way repeated measures ANOVA, F_{(24,140)}=13.01, P<0.0001. Dunnet's multiple comparison post-hoc test. n=6 per group. B. Area Under Curve (AUC) representation of 30 and 60 min data in FIG. 5, panel A.

FIG. 6 shows that repeated administration prolongs high molecular weight hyaluronan induced anti-hyperalgesia. A. Male rats received oxaliplatin (2 mg/kg, i.v.) on day 0. On day 7, 500-1200 kDa hyaluronan (2 μg/μL in a volume of 30 μL) or DMSO vehicle (30 μL) was administered topically on the dorsum of the hind paw, at the site of nociceptive threshold testing. Once the anti-hyperalgesic effect of hyaluronan wore off, hyaluronan was again administered, at the same site. Mechanical nociceptive threshold was evaluated before oxaliplatin, 7 days after its administration, and 30, 60 and 120 min after the first topical administration of 500-1200 kDa hyaluronan, and again 30, 60, 120, 180 and 240 min after the second topical administration of hyaluronan. Rats treated with oxaliplatin showed a prolongation of hyaluronan-induced anti-hyperalgesia after a second administration (two-way repeated measures ANOVA, F_{(1,5)}=138.0, P<0.0001). n=6 per group. B. Male rats received paclitaxel (1 mg/kg, i.p. every other day for a total of 4 doses). On day 7 hyaluronan (2 μg/μL in a volume of 30 μL) or DMSO vehicle (30 μL) was administered topically on the dorsum of the hind paw, at the site of nociceptive threshold testing. Once the anti-hyperalgesic effect of hyaluronan wore off, another dose of hyaluronan was administered at the site of nociceptive threshold testing. Mechanical nociceptive threshold was evaluated before paclitaxel, 7 days after its administration, and 30, 60 and 120 min after the first topical administration of hyaluronan, and again 30, 60, 120, 180 and 240 min after the second topical administration of hyaluronan. Paclitaxel-treated rats showed a prolongation of 500-1200 kDa hyaluronan-induced anti-hyperalgesia after the second administration, (two-way repeated measures ANOVA, F_{(1,5)}=29.0, P=0.003). n=6 per group.

FIG. 7 shows the effect of hyaluronan molecular weight and its combination with diverse transdermal drug delivery enhancers on the anti-hyperalgesic effect of topical hyaluronan. A. Male rats received oxaliplatin (2 mg/kg, i.v.) on day 0. On day 7, hyaluronan was administered, at 5 different molecular weight ranges (75-120, 150-300, 300-500, 500-1200, and 1500-1750 kDa), on the dorsum of the hind paw, at the site of nociceptive threshold testing, in separate groups of rats. Mechanical nociceptive threshold was evaluated before, 7 days after administration of oxaliplatin, and again 10, 30, 60 and 120 min after topical hyaluronan or vehicle. Oxaliplatin decreased mechanical nociceptive threshold (i.e., produced hyperalgesia, paired Student's t-test, baseline versus post-oxaliplatin, before hyaluronan, for each group: Vehicle P=0.003956). Topical administration of all molecular weight ranges of hyaluronan, except 70-120 kDa, produced robust anti-hyperalgesia, of similar magnitude (70-120 kDa P=0.000526, 150-300 kDa P=0.000122, 300-500 kDa P=0.000009, 500-1200 kDa P=0.000169, 1500-1750 kDa P=0.000026). The anti-hyperalgesia induced by all molecular weight ranges of hyaluronan was significant 60 min post-administration. (two-way repeated measures ANOVA, F_{(5,30)}=20.60, P<0.0001). n=6 per group. B. Male rats received oxaliplatin (2 mg/kg, i.v.) on day 0. On day 7 after oxaliplatin hyaluronan combined with protamine (2 μg/μL in a volume of 30 μL) or protamine alone as a control (30 μL) was administered topically on the dorsum of the hind paw, at the site of nociceptive threshold testing. Mechanical nociceptive threshold was evaluated before, 7 days after administration of oxaliplatin, and again 10, 30, 60 and 120 min after topical application. Topical administration of hyaluronan combined with protamine produced robust anti-hyperalgesia lasting 120 min post-administration. (two-way repeated measures ANOVA, F_{(4,40)}=24.50, P<0.0001). n=6 per group. C. Male rats received oxaliplatin (2 mg/kg, i.v.) on day 0. On day 7 after oxaliplatin hyaluronan combined with terpene (2 μg/μL in a volume of 30 μL) or terpene alone as a control (30 μL) was administered topically on the dorsum of the hind paw, at the site of nociceptive threshold testing. Mechanical nociceptive threshold was evaluated before, 7 days after administration of oxaliplatin, and again 10, 30, 60 and 120 min after topical application. Topical administration of hyaluronan combined with terpene also produced anti-hyperalgesia (two-way repeated measures ANOVA, F_{(4,40)}=41.02, P<0.0001). n=6 per group. D. Male rats received paclitaxel (1 mg/kg, i.p. every other day for a total of 4 doses). On day 7 hyaluronan combined with protamine (2 μg/μL in a volume of 30 μL) or protamine alone as a control (30 μL) was administered topically on the dorsum of the hind paw, at the site of nociceptive threshold testing in the end of the day. On the next day, 17 hours after first HWMH administration, another dose of hyaluronan was administered at the site of nociceptive threshold testing. Mechanical nociceptive threshold was evaluated before paclitaxel, 7 days after its administration, and 30, 60, 120, 180, 240, 300 and 360 min after the second topical administration of hyaluronan. Paclitaxel-treated rats showed a prolongation of 500-1200 kDa hyaluronan-induced anti-hyperalgesia after the second administration, (two-way repeated measures ANOVA, F_{(9,90)}=21.12, P<0.0001). n=6 per group.

FIG. 8 shows that topical hyaluronan-induced anti-hyperalgesia is CD44 dependent. A. Male rats received oxaliplatin (2 mg/kg, i.v.) on day 0. Starting four days later, they were treated intrathecally, (i.t.) with ODN antisense or mismatch for CD44 mRNA (120 μg/20 μL, i.t.), daily for 3 days. On day 7, approximately 24 h after the last dose of ODN, 500-1200 kDa hyaluronan (2 μg/μL in a volume of 30 μL) or its DMSO vehicle (30 μL) was administered topically on the dorsum of the hind paw, at the site of nociceptive threshold testing. Mechanical nociceptive threshold was evaluated before oxaliplatin and 7 days after its administration, and again 30, 60 and 120 min after topical hyaluronan. Results are presented as mechanical nociceptive threshold in grams. Oxaliplatin decreased mechanical nociceptive threshold, measured 4 days after its administration, in both CD44 antisense- (one-way repeated measures ANOVA F_{(100,501)}=41.13, P=0.0014) and mismatch- (one-way repeated measures ANOVA F_{(130,651)}=48.95, P=0.0002) treated groups. However, 500-1200 kDa hyaluronan only attenuated the hyperalgesia induced by oxaliplatin in the CD44 mismatch-treated group (two-way repeated measures ANOVA F_{(1,10)}=40.72, P<0.0001). n=6 per group. B. Male rats received paclitaxel (1 mg/kg, i.p., every other day for a total of 4 doses). Four days after the 1st paclitaxel injection, rats were treated with ODN antisense or mismatch (120 μg/20 μL, i.t.) for CD44 mRNA, daily for 3 consecutive days. On day 7, approximately 24 h after the last ODN dose, and the last administration of paclitaxel, hyaluronan (2 μg/μL in a volume of 30 μL) or DMSO vehicle (30 μL) was applied topically on the dorsum of the hind paw, at the site of nociceptive threshold testing. Mechanical nociceptive threshold was evaluated before paclitaxel, 7 days after its administration, and again 30, 60 and 120 min after topical 500-1200 kDa hyaluronan. Hyaluronan attenuated the hyperalgesia induced by paclitaxel only in the CD44 mismatch-treated group (two-way repeated measures ANOVA F_{(1,10)}=54.02, P<0.0001). n=6 per group.

FIG. 9 shows that topical hyaluronan attenuates itch behavior in rats.

FIG. 10 provides additional data showing that topical hyaluronan attenuates 5-HT-induced scratching behavior.

### DETAILED DESCRIPTION

The scope of the invention encompasses novel methods of using extant compositions to treat somatosensory conditions, as well as novel compositions of matter that may be applied in such applications. The various elements of the inventions disclosed herein are described next.

### Therapeutically Effective Hyaluronic Acid Compositions

Hyaluronic acid is a polymeric molecule, comprising linear repeating linked units of a disaccharide comprising a N-acetyl-D-glucosamine residue and a D-glucuronic acid, linked by alternating β-1,4 and β-1,3 glycosidic bonds. The disaccharide subunit of a hyaluronic acid polymer is as follows:

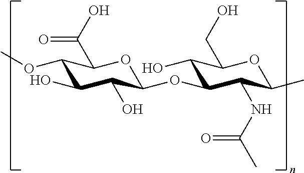

Hyaluronic acid polymers are typically described with reference to the molecular weight thereof. “Molecular weight,” as used herein encompasses the meanings thereof as known in the art. With reference to a hyaluronic acid composition comprising a particular molecular weight, the enumerated number is understood to refer to the average molecular weight of the hyaluronic acid polymers in the composition. In one embodiment, the reference to molecular weight is a number average molecular weight, often denoted M_{N}, as known in the art. In one embodiment, the reference to molecular weight is a weight average molecular weight, often referred to as M_{w}. The compositions of the invention will be understood to have some degree of polydispersity (M_{w}/M_{N}). Exemplary polydispersity values include, for example, values between 1.01 and 20.0, for example, between 1.5 and 10.0, for example, about 5.0.

In some implementations, hyaluronic acid molecular weight is recited as a range, for example hyaluronic acid having a molecular weight of 500-1200 kDa. Such references to a molecular weight range will be understood to refer to a polydisperse composition wherein the average molecular weight of the hyaluronic acid polymers therein falls within the enumerated range.

The scope of the invention encompasses the use of therapeutically effective hyaluronic acid compositions. A therapeutically effective hyaluronic acid composition comprises a hyaluronic acid composition having an enumerated molecular weight, and which has one or more selected biological or therapeutic effects, for example, in one embodiment having an anti-hyperalgesia effect and in one embodiment having an anti-pruritic effect.

In certain implementations of the invention, a therapeutically effective hyaluronic acid composition is a hyaluronic acid composition having a molecular weight of at least 90 KDa, at least 100 kDa, at least 110 KDa, at least 120 kDa, or at least 130 KDa. In various implementations, the hyaluronic acid polymers may have a molecular weight in the range of: 120-2,000 KDa; 120-1,750 KDa, 150-300 KDa, 150-500 KDa, 120-1,200 KDa, or 150-1,200 KDa. As demonstrated herein, hyaluronic acid having a molecular weight of 1500-1750 KDa is therapeutically effective. Larger polymers may be used as well.

### Topical Hyaluronic Acid Compositions for the Treatment of Somatosensory Disorders

The inventor of the present disclosure has advantageously determined that treatment of pain, e.g. neuropathic pain, inflammatory pain and the treatment of pruritis may be achieved by the novel use of topical application of hyaluronic acid. To achieve the desired therapeutic effect, the topically applied hyaluronic acid must traverse the epidermis to access and act upon nociceptors or pruriceptors present in the dermis. Hyaluronic acid having the therapeutically effective molecular weight is itself much too large and much too hydrophilic to passively traverse the epidermis. Accordingly, in the topical treatment methods of the invention, the hyaluronic acid polymers are delivered in a topical composition. The topical composition comprises:
hyaluronic acid having a therapeutically effective molecular weight; and
one or more transdermal delivery agents.

The transdermal delivery agent, as used herein comprises a material that will facilitate transit of the hyaluronic acid across the epidermis to target nociceptors of the dermis. Any number of surfactants, solvents, skin penetrating agents, and other compositions known in the art for transdermal delivery may be utilized.

In one implementation, the transdermal delivery agent, will synergize or otherwise improve the efficacy of the delivered hyaluronic acid. In such implementations, the transdermal may be referred to herein as a “transdermal drug delivery enhancer.” In one embodiment, the transdermal delivery agent is protamine, as described below, wherein it acts as a transdermal drug delivery enhancer by extending anti-pruritic and anti-hyperalgesic effects of delivered hyaluronic acids. In some embodiments, the transdermal drug delivery enhancer is DMSO or terpenes, for example, ginkolides.

DMSO. In a primary embodiment, the transdermal delivery agent is dimethyl sulfoxide (DMSO). DMSO is known in the art as an effective agent for facilitating transdermal delivery of various agents. In one embodiment, the scope of the invention comprises a pharmaceutical composition comprising:
a solution of hyaluronic acid in a solvent; and DMSO.

In this pharmaceutical composition, the solution comprises hyaluronic acid dissolved in water or another compatible (e.g., polar) solvent, for example, a solution of hyaluronic acid comprising 5-20 μg/μL hyaluronic acid in water or other solvent, for example, in some embodiments, a saturated or near saturated solution. In one embodiment, the solution comprises about 10 μg/μL. Next, the solution is combined with the DMSO by mixing. In one embodiment, the DMSO is present at a final concentration between 20 and 75% by volume, for example, a concentration of about 70-80%, for example, about 75%, with concentration percentages being expressed as percent DMSO volume in the total volume comprising the solution of dissolved hyaluronic acid and the DMSO. In some embodiments, in the final pharmaceutical composition, the concentration of hyaluronic acid is between 1.0 to 10.0 μg/μl, for example, about 1.0, about 2.0, about 3.0, about 4.0, about 5.0, about 6.0, about 7.0, about 8.0, about 9.0, or about 10.0 μg/μl.

Results presented herein demonstrate that in some implementations, concentrations of hyaluronan less than or equal to 15% of DMSO did not show high molecular weight hyaluronan-induced anti-hyperalgesia. Thus, in some implementations, a minimum DMSO concentration of about 20% (about, as used herein being, +/−3%, 5% or 10% of an enumerated value) is necessary for HMWH to penetrate the stratum corneum and produce its anti-hyperalgesic or anti-pruritic effects.

In some implementations, the composition comprises a solution of high molecular weight hyaluronan combined with DMSO at a concentration, by volume of greater than 15% DMSO, for example, at least 20% DMSO. The solution may comprise hyaluronan at the selected molecular weight dissolved in a solvent, for example, water. The solvent may comprise, for example, a concentration of hyaluronan in solvent about or equivalent to 0.5, 0.75, 1.0, 10.0 1.25, 1.5, 1.75, 2, 2.25, 2.5, 3.0, 3.25 or 3.5 μg hyaluronan per 30 μL solvent, for example, 0.0167, 0.025, 0.033, 0.01467, 0.05, 0.05833, 0.0667, 0.0833, 0.0917, 0.10, 0.1083, or 0.1167 μg hyaluronan per μL solvent.

The solution is then combined with DMSO (or, as described below, other transdermal carriers) at a selected percentage volume DMSO (e.g. [volume DMSO divided by total volume of DMSO+hyaluronan solution]×100), for example, at least 15% DMSO, greater than 15% DMSO, or at least 16%, 17%, 18%, 19%, 20%, 21%, 22%, 23%, 24%, 25%, 30%, 35%, 40%, 45%, 50%, or greater % DMSO.

In one implementation, the composition is defined as having a ratio of hyaluronan mass to DMSO volume (lower ratios meaning more DMSO). For example, a composition comprising 85% hyaluronan solvent comprising 2 μg/μL in a volume of 30 μL, and 15% DMSO will comprise 0.06667 μg/μL solvent and a mass of 0.5695 μg hyaluronan per 15 μL DMSO, for a ratio of about 0.0378 μg hyaluronan per μL DMSO. In various embodiments, the ratio of hyaluronan to DMSO is less than or equal to about 0.0378 μg hyaluronan per μL DMSO, less than or equal to about 0.035 μg hyaluronan per μL DMSO, less than or equal to about 0.030 hyaluronan per μL DMSO, less than or equal to about 0.02677 μg hyaluronan per μL [equivalent to a composition comprising 20% DMSO combined with 2 μg hyaluronan/30 μL solvent) less than or equal to about 0.025 μg hyaluronan per μL DMSO, less than or equal to about 0.024 hyaluronan per μL DMSO, less than or equal to about 0.023 μg hyaluronan per μL DMSO, less than or equal to about 0.022 μg hyaluronan per μL DMSO, less than or equal to about 0.021 hyaluronan per μL DMSO, less than or equal to about 0.020 μg hyaluronan per μL DMSO or lower.

Other Transdermal Delivery Agents. Any transdermal delivery agent known in the art may be used in the topical hyaluronic acid compositions of the invention. Exemplary transdermal delivery agents include:
Protamine and variants thereof;
Terpenes, for example: thymol, tetra-hydrogeraniol, pulegone; nerolidol; menthol; linalool; farnesol; carvone; camphor; and anethole;
Pyrrolidones, for example: N-methylpyrrolidone; Poly[acrylonitrile-co-(N-vinyl pyrrolidone)]; and 2-pyrrolidone;
Liposomes or other nanoparticles, such as nanoparticles comprising: chitosan; polyalkylcyanoacylates; poly-lactic acid; poly-e-caprolactone; poly-glycolic acid; and poly-lactic-co-glycolic acid (PLGA);
Skin penetrating peptides, such as Peptide R7 (Polyarginine-7); YARA (YARAAAR-QARA (SEQ ID NO: 1)); WLR (WLRRIKAWLRRIKAWLRRIKA (SEQ ID NO: 2)); TAT (YGRKKRRQRRR (SEQ ID NO: 3)); Penetratin (RQIKIYFQNRRMKWKK (SEQ ID NO: 4)); VP22 (DAATATRGRSAASRPTERPRAPARSASRPRRPVE (SEQ ID NO: 5)); Membrane translocating sequence peptide (AAVALLPAVLLALLAP (SEQ ID NO: 6)); Transportan (GWTLNSAGYLLKINLKALAALAKKIL (SEQ ID NO: 7)); Polyarginine (RRRRRRRRRRR (SEQ ID NO: 15)); Model amphipathic peptide (KLALKLALKALKAALKLA (SEQ ID NO: 8)); Flock house virus (RRRRNRTRRNRRRVR (SEQ ID NO: 9)); and Pep-1 (hydrophobic/NLS) (KETWWETWWTEWSQPKKKRKV (SEQ ID NO: 10));
other arginine oligomers, including Rothbard et al., 2000, Conjugation of arginine oligomers to cyclosporin A facilitates topical delivery and inhibition of inflammation: Nature Medicine 6:1253-1257;
long-chain fatty acids, such as: oleic acid; lauric acid; myristic acid; and capric acid;
glycols, such as diethylene glycol and tetraethylene glycol;
surfactants, such as: polyoxyethylene-2-oleyl ether; and polyoxy ethylene-2-stearly ether;
essential oils, such as oils of: eucalyptus; Chenopodium; and ylang-ylang; and
other transdermal penetration agents, such as: 4-decyloxazolidin-2-one; azone; urea, and nicotinamide.

In one embodiment, the topical hyaluronic acid formulation comprises hyaluronic acid in combination with a nanoparticulate carrier as described in: Tokudome et al., 2018. A new strategy for the passive skin delivery of nanoparticulate, high molecular weight hyaluronic acid prepared by a polyion complex method, Scientific Reports 8: 2336.

Protamine. In one embodiment, the transdermal delivery agent comprises protamine. Protamines, as known in the art are small polycationic proteins. Protamines are produced in the spermatids of many species and aid in the compact packaging of DNA. The scope of the invention encompasses the use of any protamines of any species or artificial protmaines, or protamine derivative, including, for example, monoprotamines, diprotamines, triprotamines, fish protamines such as Salmine or Clupeine, protamine in free base form, protamine salts such as protamine sulfate and protamine chloride, low-molecular-weight protamine forms, polyethylene glycol (PEG)-conjugated protamines, and Mannosylated protamine sulfate.

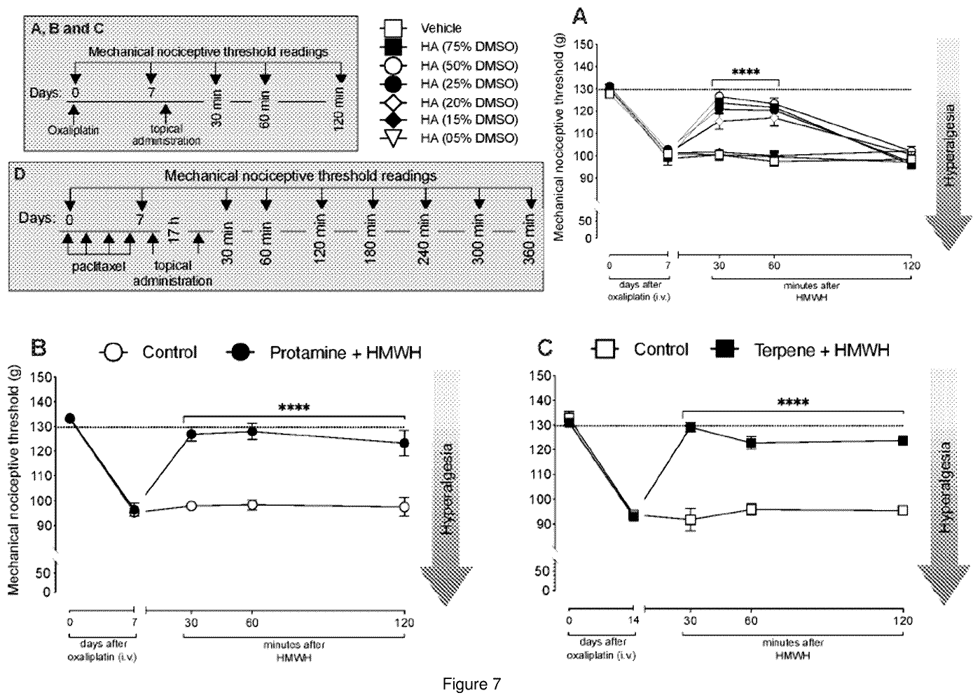

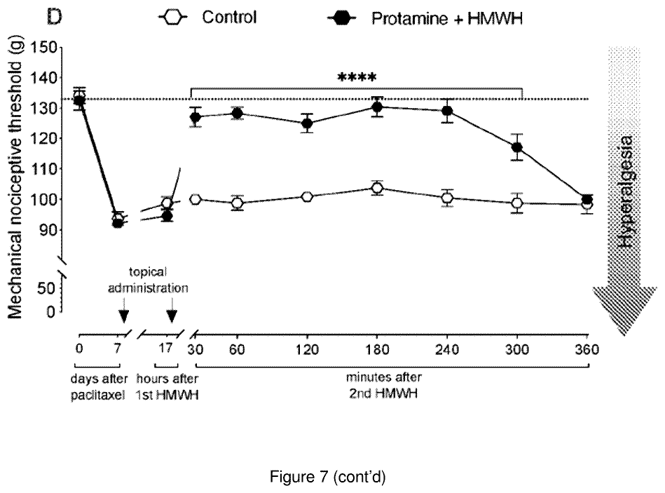

In the formulations and method of the invention, protamine may be combined with hyaluronic acid by any process. For example, as described in the Examples, Hyaluronan combined with protamine was prepared as follows: A stock solution of Hyaluronan was made by dissolving Hyaluronans in distilled water at a concentration of 10 μg/μL. Protamine was first dissolved in distilled water (dH₂O) to a concentration of 5 μg/μl, then stock solution of hyaluronan was combined with the protamine solution at the final concentration (2 μg/μl in a volume of 3 μl, administered topically). Topical administration of hyaluronan combined with protamine transdermal delivery agent produced robust anti-hyperalgesia, for example, as depicted in FIG. 7B.

Additional hyaluronic acid protamine formulations include those described in: Tokudome et al. (2018) A new strategy for the passive skin delivery of nanoparticulate, high molecular weight hyaluronic acid prepared by a polyion complex method. Sci Rep 8:2336, for example, comprising mixtures of hyaluronic acid and protomine ranging from 10:90, 20:80, 30:70, 40:60, 50:50; 40:60, 70:30, 80:20 and 90:10 hyaluronic acid to protamine by weight.

Terpenes, particularly Ginkolides. In one implementation, the transdermal delivery agent is a terpene. In one embodiment, the terpenes are terpenes derived from Ginko spp. In various embodiments, the terpenes comprise one or more of:

bilobalide:

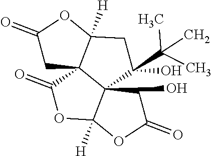 [SMILES: CC(C)(C)[C@]1(O)C[C@@H]2OC(=O)C[C@@]23C(=O)O[C@@H]2OC(=O)[C@H](O)[C@]213]

### Ginkolide A:

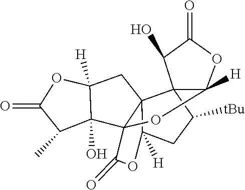 [SMILES: C[C@@H]1C(=O)O[C@H]2CC34[C@H]5C[C@@H](C(C)(C)C)C36[C@H](OC(=O)[C@@H]6O)OC4(C(=O)O5)[C@]21O]

### Ginkolide B:

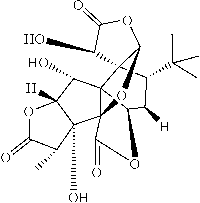

### Ginkolide C:

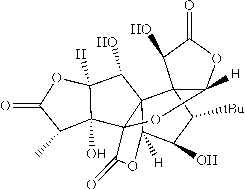 [SMILES: C[C@@H]1C(=O)O[C@H]2[C@H](O)C34[C@@H]5OC(=O)C3(O[C@H]3OC(=O)[C@H](O)C34[C@H](C(C)(C)C)[C@H]5O)[C@@]12O]

### and Ginkolide J:

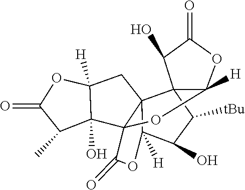 [SMILES: C[C@@H]1C(=O)O[C@H]2CC34[C@@H]5OC(=O)C3(O[C@H]3OC(=O)[C@H](O)C34[C@H](C(C)(C)C)[C@H]5O)[C@]21O]

Terpenes may be utilized the transdermal carrier, as known in the art, for example, as described in Chen et al. (2016) Natural Terpenes as Penetration Enhancers for Transdermal Drug Delivery. Molecules 21: 1709.

In the formulations and method of the invention, terpenes, e.g. ginkolides, may be combined with hyaluronic acid by any process. For example, as described in the Examples, terpenes comprising a mixture of ginkolides was first dissolved in dH₂O at a concentration of 2 μg/μl, then hyaluronan was dissolved in this stock solution of terpene to its final concentration (2 μg/μl in a volume of 3 μl, administered topically). Topical administration of hyaluronan with ginkilides as the transdermal delivery agent produced anti-hyperalgesia, as depicted in FIG. 7C.

### Formulations and Dosage forms

The topical hyaluronic acid composition of the invention may comprise any formulation capable of effective topical application. Exemplary dosage forms include creams, salves, ointments, drug delivery patches, and other dosage forms known in the art for topical delivery of agents.

It will be understood that the topical hyaluronic acid compositions of the invention, in addition to comprising hyaluronic acid and one or more transdermal delivery agents, may further include any other constituents, for example, carriers, timed-release compositions, fillers, excipients, coloring agents, scents, and other compositions of matter. The topical hyaluronic acid compositions of the invention may further comprise combination products, for example, including additional active agents, such as topical anesthetics, anti-inflammatory agents, moisturizers, sunscreen, and other agents that augment or supplement the therapeutic properties of the compositions.

### Methods of the Invention

Treatment of pain. The scope of the invention encompasses a general method as follows:
A method of treating pain in a subject in need of treatment for a pain condition by the administration to the subject of a therapeutically effective amount of hyaluronic acid having a therapeutically effective molecular weight.

The pain condition may be any condition wherein hyaluronic acid-mediated signaling in nociceptors in the skin will alleviate the condition. Alleviation, as used herein, encompasses any reduction in pain, hyperalgesia or allodynia, reduction in the perception of pain, inhibition of processes which underlie the condition; or any other therapeutic effect with regards to the enumerated condition. The pain to be alleviated may be of any type, including neuropathic, nociceptive, inflammatory, stress-related, psychogenic, or functional pain.

In a primary implementation, the pain condition is neuropathic pain. Exemplary forms of neuropathic pain include, for example: chemotherapy-induced neuropathic pain; peripheral neuropathy; focal neuropathy, autonomic neuropathy; proximal neuropathy, diabetic neuropathy, trigeminal neuralgia; small-fiber neuropathy, and compression mononeuropathy. Other forms of neuropathy include CRPS-1, CRPS-2, drug-induced, infectious, alcohol-induced, idiopathic and traumatic neuropathy.

In another implementation, the pain condition is inflammatory pain, i.e., pain associated with inflammation or reparatory processes in response to injury, for example, pain associated with peripheral sensitization.

In other embodiments, the pain condition is a condition resulting from trauma, injury, surgery, disease, allergy, infection, or other pain-causing factors. Exemplary conditions include: stress-related pain: fibromyalgia syndrome, pain associated with irritable bowel syndrome, pain associated with post-traumatic stress-disorder (PTSD), and dry eye syndrome.

The subject may be a human subject, or may be any non-human animal, such as a test animal, pet, livestock, or veterinary subject.

The administration will be in a therapeutically effective amount, meaning an amount sufficient to have any measurable therapeutic effect. For example, in one implementation, the topical administration comprises the administration of 1.0 μg to 1.0 gram, for example, about 1 μg, about 10 μg, about 50 μg, about 100 μg, about 250 μg, about 500 μg, about 750 μg, about 1 mg, about 10 mg, about 50 mg, about 100 mg, about 250 mg, about 500 mg, about 1 g; hyaluronic acid per dose, for example 1 μg-100 μg hyaluronic acid per square centimeter of skin surface. Depending on dosage form, condition, and other factors, administrations may be, for example, multiple times per day, once per day, every other day, once per week, or otherwise as needed.

In a primary implementation, the administration is topical, for example, administered to the skin by exposure thereto. Exemplary administrations include the application, for example, by hand, of lotions, ointments, salves, creams or other like dosage forms or topical application by a patch (e.g. adhesive patch) or dressing coated or saturated with an hyaluronic acid formulation. Administration will generally be localized to the area where pain is being experienced, for example, to the hands, face, feet, limbs or other afflicted area or into body cavities, such as joints, abdomen and thorax. In some embodiments the administration is to an internal compartment of the body, for example, by injection, infusion, or other administration, for example, to body cavities: joints, the abdominal cavity, and/or into the gastrointestinal tract.

Treatment of Pruritus. The scope of the invention encompasses a general method as follows:
A method of treating pruritus in a subject in need of treatment therefor by the administration to the subject of a therapeutically effective amount of hyaluronic acid having a therapeutically effective molecular weight.

The pruritus may be any condition encompassing itch or like sensations wherein hyaluronic acid-mediated signaling in pruriceptors in the skin will alleviate the condition. Alleviation, as used herein, encompasses any reduction in itch, reduction in the perception of itch, inhibition of processes which underlie the condition; or any other therapeutic effect with regards to the enumerated condition.

The pruritus may be of any type or by any cause. Exemplary forms of pruritus include: idiopathic generalized pruritus; allergic contact dermatitis; atopic dermatitis; urticaria; pruritus associated with psoriasis; pruritus associated with dry skin; advanced age or senile pruritus; pruritus associated with sunburn; pruritus from insect bite or other biting organism; systemic pruritus; renal or uremic pruritus; cholestatic pruritus; pruritus associated with thyroid conditions; pruritus associated with cancer; pruritis associated with conditions of the eye; brachioradial pruritus; pruritus associated with shingles; pruritus ani; and psychogenic or somatoform pruritus.

In a primary implementation of the method, the administration of hyaluronic acid is topical. Topical administration is advantageously non-invasive and is amenable to convenient self-administration. In alternative implementations, the administration may be by subdermal injection, injection by microneedles, sonic-assisted delivery, and other methods of delivering hyaluronic acid of the selected molecular weight to pruriceptors wherein pruritus is being perceived.

The subject may be a human subject, or may be any non-human animal, such as a test animal, pet, livestock, or veterinary subject.

The administration will be in a therapeutically effective amount, meaning an amount sufficient to have any measurable therapeutic effect. For example, in one implementation, the topical administration comprises the administration of hyaluronic acid as disclosed above for the treatment of pain. Depending on dosage form, condition, and other factors, administrations may be, for example, multiple times per day, once per day, every other day, once per week, or otherwise as needed.

Administration will generally be localized to the area where pruritus is being experienced, for example, to the hands, face, feet, limbs, or other afflicted area.

### EXAMPLES

Example 1. Topical Administration of High Molecular Weight Hyaluronic Acid for Treatment of Pain. The manuscript appended hereto, entitled “Topical Co-application of Hyaluronan with Transdermal Drug Delivery Enhancers for the Treatment of Inflammatory and Neuropathic Pain” describes the elucidation of therapeutically effective molecular weights of hyaluronic acid and concentration of the transdermal drug delivery enhancer DMSO that may be applied in the methods of the invention. The effective topical administration of hyaluronic acid to treat pain is demonstrated.
Example 2. Administration of High Molecular Weight Hyaluronic acid for the Treatment of Pruritus. Pruritis (itch) is the unpleasant sensation that compels a person to scratch themselves. Chronic itch, which persists for 6 weeks or longer, has a lifetime prevalence in the general populations of 22% PMID: 27578064. Chronic itch produces mental distress and significantly affects the individual's quality of life PMID: 27517368, but the treatment of chronic itch has been challenging. Itch is sensed by primary afferent peripheral sensory neurons (puriceptors), and it has been hypothesized that puriceptors can also function as nociceptors PMID: 31940043.

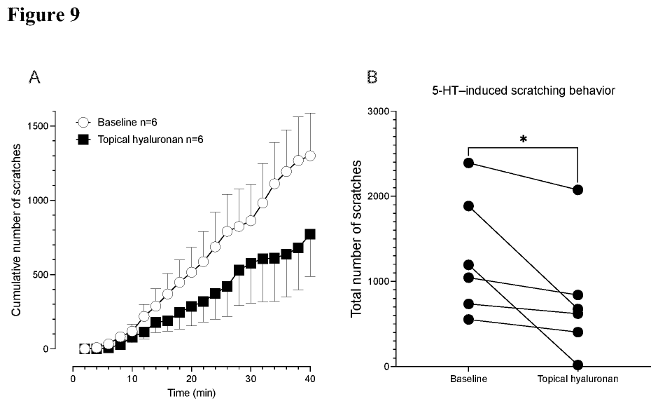

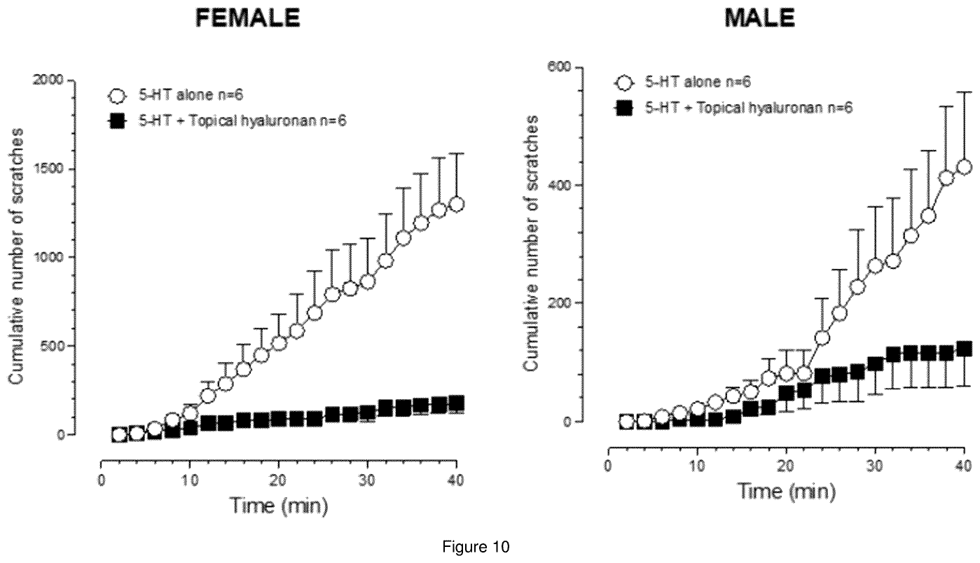

Topical hyaluronan attenuates itch behavior in rats. Intradermal administration of serotonin (5-HT) elicited acute scratching behavior, using the method of Nojima and Carstens {Nojima and Carstens, 2003, #99335; PMID: 12814838}. Rats were acclimated to a testing chamber for 30 min, and then briefly anesthetized with 2% isoflurane, during which the fur over the nape of the neck was shaved, and 5-HT (200 μg in 10 μl 0.9% saline vehicle) injected with a 30 gauge needle. In the topical hyaluronan group, hyaluronan (100 μg in 50 μl 75% DMSO in water vehicle) was applied to the skin 30 mins and again 5 mins before administration of 5-HT. Rats were placed back into testing chambers and their behavior video recorded for 40 min. Number of hind paw scratches directed at the injection site were counted by watching video playback at half normal speed, and total number of scratches in 2-min intervals recorded. Rats were treated with 5-HT alone and topical hyaluronan with 5-HT, with 4 days between experimental sessions, allowing for use of paired t-tests to compare data. Data are presented as cumulative number of scratches (FIG. 9A), and as total scratches over the 40-min observation period (FIG. 9B). Topical hyaluronan significantly attenuated the number of 5-HT scratches (one-tailed paired Student's t-test, *P=0.0275, n=6). See also, FIG. 10, which includes additional data showing that topical hyaluronan attenuates 5-HT-induced scratching behavior. FIG. 10 shows data from experiments in which male and female rats received intradermal injections of 5-HT alone, or after topically administered 500-12200 kDa hyaluronan in protamine vehicle. Data are presented as cumulative number of scratches. FIG. 10 shows that topical hyaluronan significantly attenuated the number of 5-HT-induced scratches in both females and males (one-tailed paired Student's t-test, females: P<0.0001; males: P<0.0004), all groups n=6.

All patents, patent applications, and publications cited in this specification are herein incorporated by reference to the same extent as if each independent patent application, or publication was specifically and individually indicated to be incorporated by reference. The disclosed embodiments are presented for purposes of illustration and not limitation. While the invention has been described with reference to the described embodiments thereof, it will be appreciated by those of skill in the art that modifications can be made to the structure and elements of the invention without departing from the spirit and scope of the invention as a whole.

### Example 1

### Topical Co-Application of Hyaluronan with Transdermal Drug Delivery Enhancers for the Treatment of Inflammatory and Neuropathic Pain

### Abstract

We have previously shown that intradermal injection of high molecular weight hyaluronan (500-1200 kDa) produces localized anti-hyperalgesia in diverse preclinical models of inflammatory and neuropathic pain. In the present experiments we studied the therapeutic efficacy of topical hyaluronan combined with transdermal drug delivery enhancers, dimethyl sulfoxide (DMSO), protamine or terpene, in preclinical models of inflammatory and neuropathic pain. Topical application of 500-1200 kDa hyaluronan (the molecular weight range used in our previous studies employing intradermal administration), dissolved in 75% DMSO in saline, markedly reduced prostaglandin E2 (PGE₂) hyperalgesia, in both male and female rats. While topical 500-1200 kDa hyaluronan dose-dependently attenuated oxaliplatin and paclitaxel chemotherapy-induced peripheral neuropathy (CIPN) pain in male rats, it lacked efficacy in females. However, following ovariectomy or intrathecal administration of an oligodeoxynucleotide (ODN) antisense to G-protein coupled estrogen receptor (GPR30) mRNA, CIPN in female rats was also attenuated by topical hyaluronan. While topical administration of 150-300, 300-500 and 1500-1750 kDa hyaluronan, combined with DMSO, also attenuated CIPN, a lower molecular weight range of hyaluronan (70-120 kDa) did not. The topical administration of the combination of hyaluronan with two other skin penetration enhancers, protamine or terpene, also attenuated CIPN hyperalgesia, which was more prolonged than when using DMSO as the penetration enhancer. Our results demonstrate that topical hyaluronan, with a non-toxic skin penetration enhancer can induce anti-hyperalgesia, supports the combination of hyaluronan with transdermal enhancers as a novel pain therapy.

### Introduction

Intra-articular injection of high molecular weight hyaluronan is widely used for the treatment of pain in patients with osteoarthritis [3; 23; 28; 44; 73]. While it is generally thought to attenuate osteoarthritis pain by its viscoelastic/cushioning properties [25], high molecular weight hyaluronan has also been shown to have receptor-mediated anti-inflammatory and immunosuppressant effects [26; 39; 45; 51; 77]. High molecular weight hyaluronan can signal via plasma membrane receptors, best characterized for cluster of differentiation 44 (CD44), considered the cognate hyaluronan receptor [71; 74; 75], which is present on nociceptors [13; 34; 36]. We previously demonstrated that the attenuation of nociceptor CD44, by intrathecal (i.t.) administration of an oligodeoxynucleotide (ODN) antisense to CD44 mRNA, or the intradermal administration of a CD44 receptor antagonist, both decreased 500-1200 kDa hyaluronan-induced anti-hyperalgesia [13; 34], demonstrating that the anti-hyperalgesia induced by high molecular weight hyaluronan is mediated by its action at CD44 on nociceptors.

Neuropathic pain, a major side effect of several classes of cancer chemotherapy drugs [79], commonly referred to as chemotherapy-induced peripheral neuropathy (CIPN), is a debilitating condition for which there is currently no FDA approved treatment. CIPN is estimated to occur in ˜40% [49; 59] of the ˜17 million cancer survivors in the United States [50], emphasizing the magnitude of this problem, and bringing an urgency to understanding underlying mechanisms, for the development of effective treatments. The prevalence of CIPN is chemotherapy agent-dependent, being highest (70%-100%) for the platinum-based class of chemotherapy drugs [10; 47; 79]. Oxaliplatin, a third-generation platinum, and paclitaxel, a first-line taxane, produce pain [17] [33] that can persist for months after completion of chemotherapy [46; 56]. And, patients receiving bortezomib, a proteasome inhibitor used for the treatment of multiple myeloma and certain types of lymphoma [79], can also develop neuropathic pain (CIPN) that may last for weeks, months or even years after completion of treatment [32].

Since high molecular weight hyaluronan poorly penetrates the skin [72], in the present study we evaluated the anti-hyperalgesic effect of topical administration of hyaluronan combined with chemically diverse transdermal drug delivery enhancers, dimethyl sulfoxide (DMSO) [76], protamine [72] and terpene [21], in preclinical models of inflammatory and neuropathic (chemotherapy-induced painful peripheral neuropathy (CIPN)) pain. We report that the addition of a transdermal drug delivery enhancer is necessary for topical hyaluronan to attenuate inflammatory and neuropathic pain and establish the molecular weight range of hyaluronan and concentration range of a transdermal drug delivery enhancer needed to treat inflammatory and neuropathic pain.

### Methods

Experimental animals. Experiments were performed on 220-400 g male and female Sprague-Dawley rats, purchased from Charles River Laboratories (Hollister, CA, USA), housed three per cage, under a 12-hour light/dark cycle, in a temperature- and humidity-controlled animal care facility at the University of California, San Francisco. Food and water were available ad libitum. Experimental protocols were approved by the University of California, San Francisco, Institutional Animal Care and Use Committee, and adhered to the National Institutes of Health Guidelines for the care and use of laboratory animals.

Measurement of nociceptive threshold. Mechanical nociceptive threshold was quantified with an Ugo Basile Analgesymeter (Stoelting, Wood Dale, IL, USA), used to perform the Randall-Selitto paw-withdrawal test [61; 68; 69]. This device uses a dome-shaped plinth to apply a mechanical force that increases linearly with time, to the dorsum of a rat's hindpaw. To perform the Randall-Selitto paw-withdrawal test, rats were placed in cylindrical acrylic restrainers with lateral ports that allow access to the hind paw, as described previously [7]. Rats were acclimatized to the testing procedure by placing them in restrainers for 1 h per day for 3 consecutive daily training sessions, and for 30-40 min prior to experiments [7].

Mechanical nociceptive threshold is defined as the force, in grams, applied by the plinth to the dorsum of the hindpaw, at which a rat withdraws its paw from the stimulus. Baseline paw-withdrawal threshold is defined as the mean of three readings taken before administration of test agents. To minimize experimenter bias, individuals conducting experiments were blinded to experimental treatments. Each experiment was performed on a different group of rats. Data are presented as mechanical nociceptive threshold, in grams (g) or percentage change from preintervention baseline.

Drugs. The following drugs were used in this study: 500-1200 kDa hyaluronan purchased from Tocris (Minneapolis, MN, USA); 70-120 kDa, 150-300 kDa, 300-500 kDa, and 1500-1750 kDa hyaluronan, as well as the cancer chemotherapy agents (paclitaxel and oxaliplatin), prostaglandin (PGE₂), Cremophor EL and dimethyl sulfoxide (DMSO) and Ginkgo terpene lactones (here referred to as terpene) purchased from Sigma-Aldrich (St. Louis, MO, USA); bortezomib, purchased from LC Laboratories (Woburn, MA, USA); and, protamine sulfate from Thermo Fisher Scientific (Waltham, MA, USA).

Hyaluronans were initially dissolved in distilled water (dH₂O) at a concentration of 10 μg/μL, stock solution, and further diluted by adding DMSO with three final concentrations of hyaluronan (1, 2 and 4 μg/μL), protamine or terpene; unless otherwise stated, the final concentration of DMSO was 75%. The DMSO vehicle was 75% DMSO in 0.9% saline. In one experiment, 500-1200 kDa hyaluronan was administered topically in 0.9% saline vehicle. PGE₂ was dissolved in 0.9% saline at the same final concentration that we previously injected intradermally (100 ng/5 μL) [35]. In the experiments in which hyaluronan was dissolved in protamine or terpene vehicle, protamine was first dissolved in distilled water (dH₂O) to a concentration of 5 μg/μ1, stock solution of hyaluronan was combined with protamine at the final concentration of 2 μg/μ1, in a volume of 3 μl, for topical administration; terpene was first dissolved in dH₂O at the concentration of 2 μg/μ1, then hyaluronan was dissolved in stock solution of terpene to its final concentration of 2 μg/μ1, in a volume of 3 μl, for topical administration).

Oxaliplatin CIPN: Oxaliplatin was freshly dissolved in 0.9% saline at a concentration of 2 mg/mL just prior to its tail vein intravenous (i.v.) administration (1 mL/kg), to rats briefly anesthetized with isoflurane (2.5% in O₂).

Paclitaxel CIPN: Paclitaxel was dissolved in Cremophor EL and ethanol (1:1) [1; 6; 20; 22] and diluted in 0.9% saline to a concentration of 1 mg/mL, just prior to its intraperitoneal (i.p.) administration (1 mg/kg, i.p.) [19; 42] to rats briefly anesthetized with isoflurane (2.5% in O₂), every other day for a total of 4 doses.

Bortezomib CIPN: Bortezomib was dissolved in 3% DMSO and 97% saline, and diluted in 0.9% saline, to a concentration of 1 mg/mL, just prior to intravenous (0.2 mg/kg, i.v.) administration (1 mL/kg), via tail vein injection, to rats briefly anesthetized with isoflurane (2.5% in O₂), every other day for a total of 4 doses.

Hyaluronans (30 μL) were administered topically on the dorsum of the hind paw, dispensed from a P200 pipette (Gilson, Middleton, WI, USA) with a plastic pipette tip and then spread manually.

Antisense oligodeoxynucleotides (ODNs). The roles of nociceptor CD44 and GPR30 in high molecular weight hyaluronan-induced anti-hyperalgesia were assessed by intrathecal (i.t.) administration of ODN antisense against unique regions of the rat mRNA sequences for CD44 and GPR30, respectively.

Antisense ODN sequences used in these experiments were:

CD44 ODN antisense:
5′-GAA AAG GGT CGC GGG GG-3′
(GenBank accession number NM_012924.2;
SEQ ID NO: 11)
GPR30 ODN antisense:
5′-ATG TTC AGA GAG GTC CCC AG-3′
(GenBank accession number NM_133573;
SEQ ID NO: 12)

Mismatch ODN, corresponding to the antisense sequence with mismatched bases (denoted by bold letters), have no sequence homologies in the rat gene database. Mismatch ODN sequences were:

CD44 ODN mismatch:
(SEQ ID NO: 13)
5′-CCC CCG CGA CCC TTT TC-3′
GPR30 ODN mismatch:
(SEQ ID NO: 14)
5′-AGG TCC AGA AAG ATG CCA AG-3′

We have previously shown that these two antisense ODN sequences, when compared to rats treated with mismatch ODN, synthesized by Life Technologies (Carlsbad, CA, USA), decrease CD44 and GPR30 [4] protein in rat dorsal root ganglia.

Before use, ODNs were reconstituted in nuclease-free 0.9% saline and then administered by the intrathecal (i.t.) route. As described previously [2], rats were anesthetized with isoflurane (2.5% in O₂) and 120 μg of ODN, in a volume of 20 μL, injected i.t., using a syringe (300 units/μL) attached to a 29-gauge hypodermic needle that was inserted into the subarachnoid space, between the L4 and L5 vertebrae. The i.t. site of the injectate was confirmed by a sudden flick of the rat's tail, a reflex evoked by subarachnoid space access and bolus i.t. injection [48]. Animals regained consciousness approximately 2 minutes after i.t. injection of antisense or mismatch ODN. The use of antisense ODN, administered i.t., to attenuate the expression of proteins essential for their role in nociceptor sensitization, is well supported by previous studies, by others [54; 60; 64; 66; 67], as well as our group [5; 7; 8; 11; 34-36; 58].

Ovariectomy. Ovariectomy was performed on 23-26 day old (i.e., prepubertal) female rats that were used for behavioral experiments 3 weeks later (i.e., as adults) [41]. To perform ovariectomy, animals were anesthetized with isoflurane (3% in oxygen) and received preoperative meloxicam (˜5 mg/kg, s.c.) and bupivacaine (˜0.1 mg/kg s.c. at the incision site) for perioperative pain control. Briefly, ovaries were accessed through bilateral cutaneous followed by peritoneal incisions. Once the ovaries were located, their vascular bundles were ligatured with 5-0 silk suture (Perma-Hand Silk® Ethicon, Johnson & Johnson, Somerville, NJ). Ovaries were then excised, and the peritoneal and cutaneous incisions closed with 5-0 silk suture.

Statistical analysis. Data from behavioral experiments are presented as mechanical nociceptive threshold in grams or percentage change from pre intervention baseline nociceptive threshold. Experiments were performed with the experimenter blinded to experimental groups. Repeated-measures one-way and two-way ANOVAs or Student's t-test was used for data analysis. Prism 9.3 (Graph Pad Software) was used to generate graphics and to perform statistical analyses; P<0.05 is considered statistically significant. Data are presented as mean±SEM.

### Results

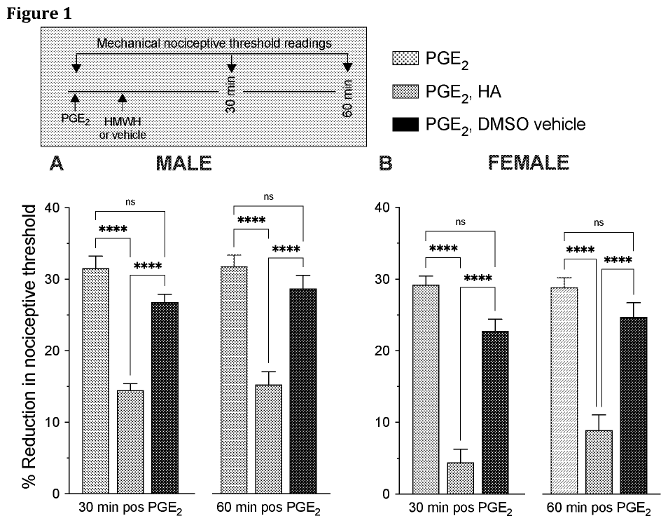

Hyaluronan in DMSO vehicle produces anti-hyperalgesia for inflammatory pain. When PGE₂ (100 ng) was injected intradermally, at the site of nociceptive testing on the dorsum of the hind paw, in male and female rats, it produced robust mechanical hyperalgesia; and, when 10 min later 500-1200 kDa hyaluronan (2 μg/μL, 30 μL) dissolved in DMSO was administered topically, at the same site, PGE₂ hyperalgesia was reversed, in both male (FIG. 1A) and female (FIG. 1B) rats. Of note, topical administration of 500-1200 kDa hyaluronan combined with the transdermal drug delivery enhancer, DMSO, produced anti-hyperalgesia of similar magnitude to that produced by its intradermal injection [13].

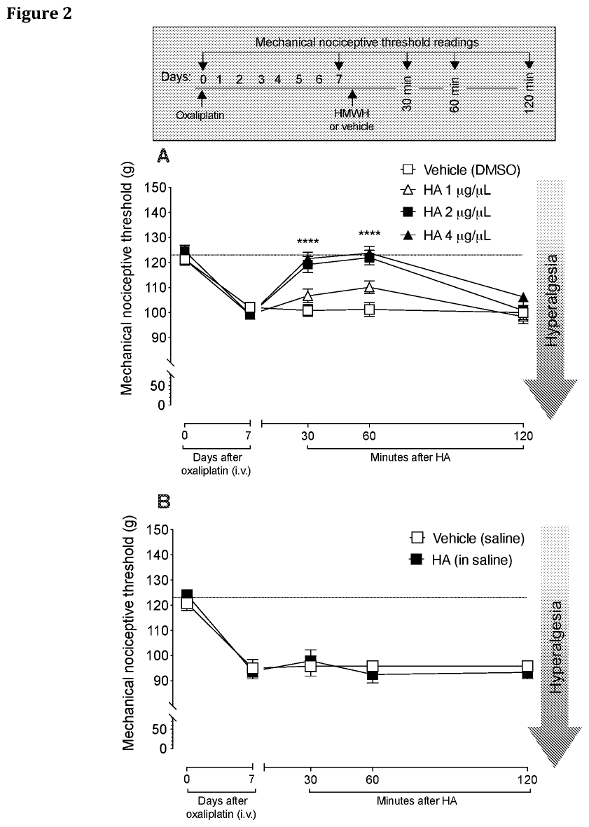

Topical hyaluronan induces dose-dependent anti-hyperalgesia for neuropathic pain in male and gonadectomized female rats. We evaluated the anti-hyperalgesia induced by hyaluronan in preclinical models of neuropathic pain, and its dose dependence, for the effect of topical 500-1200 kDa hyaluronan in DMSO, in male rats with oxaliplatin CIPN. Rats that received oxaliplatin (1 mL/kg, i.v.) developed long-lasting hyperalgesia (FIG. 2A). Seven days after oxaliplatin they received 500-1200 kDa hyaluronan in DMSO, at one of three concentrations (1, 2 or 4 μg/μL), each in a volume of 30 μL, applied topically on the dorsum of the hind paw, at the site of nociceptive threshold testing. While topical administration of 500-1200 kDa hyaluronan, 1 μg/μL, only attenuates CIPN hyperalgesia 10 min after application, 2 and 4 μg/μL attenuated hyperalgesia 10, 30 and 60 min after application. The anti-hyperalgesic effect of 2 and 4 μg/μL 500-1200 kDa hyaluronan dissipated by 120 min after application (FIG. 2A). When 500-1200 kDa hyaluronan (2 μg/μL) was dissolved in saline, without a transdermal drug delivery enhancer (i.e., 0.9% saline, without DMSO) and applied topically, it did not attenuate oxaliplatin CIPN hyperalgesia (FIG. 2B).

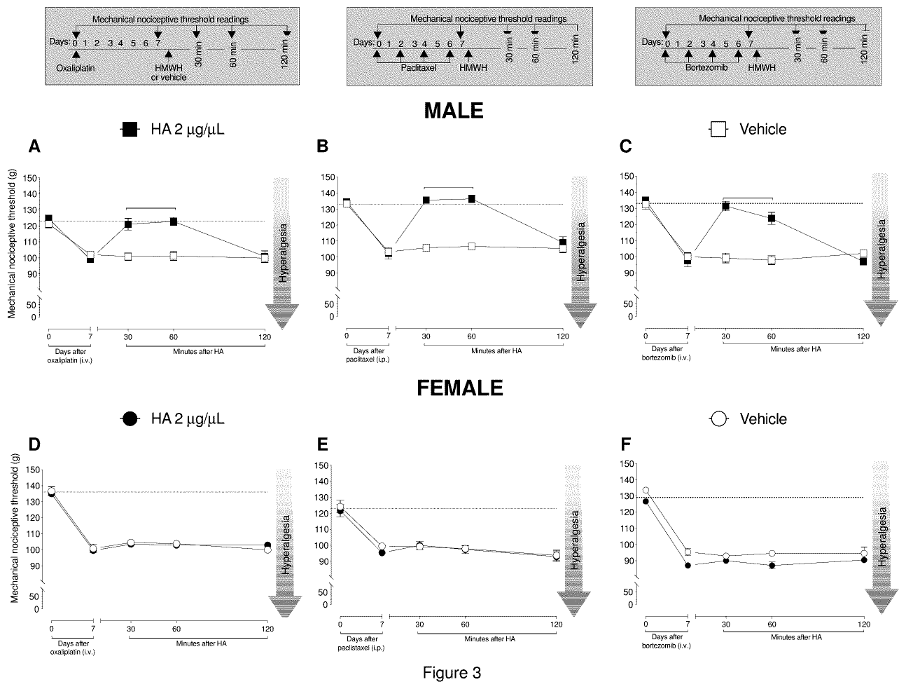

We next compared the anti-hyperalgesia induced by 500-1200 kDa hyaluronan in male and female rats with paclitaxel, oxaliplatin and bortezomib CIPN. To generate oxaliplatin CIPN, male (FIG. 3A) rats received oxaliplatin (2 mg/kg, i.v.). Seven days later, at which time rats demonstrated robust mechanical hyperalgesia, they received 500-1200 kDa hyaluronan (2 μg/μL, 30 μL) or DMSO vehicle, administered topically on the dorsum of the hind paw. In male rats with oxaliplatin CIPN, 500-1200 kDa hyaluronan induced anti-hyperalgesia (FIG. 3A). A separate group of male rats (FIG. 3B) were treated with paclitaxel, every other day for 4 days. Seven days after the first dose of paclitaxel, they received 500-1200 kDa hyaluronan (2 μg/μL, 30 μL) or DMSO vehicle, administered topically on the dorsum of the hind paw. An additional group of male rats (FIG. 3C) received bortezomib (0.2 mg/kg, i.v., every other day for a total of 4 doses). Approximately 24 h after the last dose of bortezomib, 500-1200 kDa hyaluronan (2 μg/μL, 30 μL) or DMSO vehicle was applied at the site of nociceptive threshold testing. Anti-hyperalgesia induced by topical administration of 500-1200 kDa hyaluronan was observed in male rats with paclitaxel-induced CIPN. In female rats topical administration of 500-1200 kDa hyaluronan, in DMSO vehicle, did not attenuate hyperalgesia induced by oxaliplatin (FIG. 3D), paclitaxel (FIG. 3E) or bortezomib (FIG. 3F).

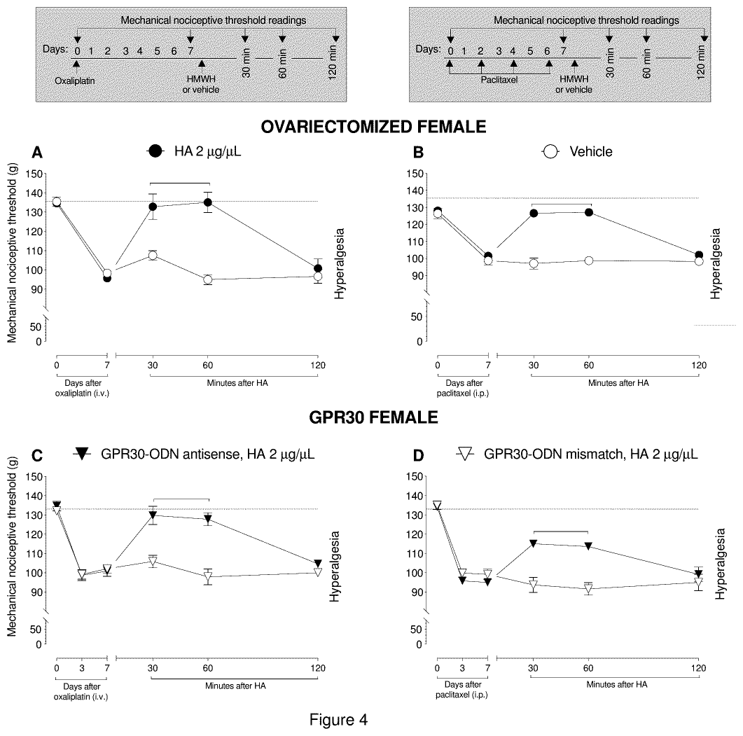

To determine if there is a sex hormone-dependence to the lack of effect of topical 500-1200 kDa hyaluronan in female rats with CIPN, we performed ovariectomy 3 weeks prior to treating female rats with paclitaxel (FIG. 4A) or oxaliplatin (FIG. 4B). In ovariectomized female rats 500-1200 kDa hyaluronan induced anti-hyperalgesia of similar magnitude to that observed in gonad intact male rats with paclitaxel- and oxaliplatin-induced CIPN. To determine if this effect of sex hormones on 500-1200 kDa hyaluronan-induced anti-hyperalgesia to CIPN was due to their action on sensory neurons, female rats with paclitaxel (FIG. 4C) and oxaliplatin (FIG. 4D) CIPN were treated with i.t. ODN antisense to GPR30 mRNA for 3 consecutive days. As for ovariectomized females, rats with oxaliplatin and paclitaxel CIPN that received GPR30 antisense, also demonstrated 500-1200 kDa hyaluronan-induced anti-hyperalgesia (FIGS. 4C and 4D).

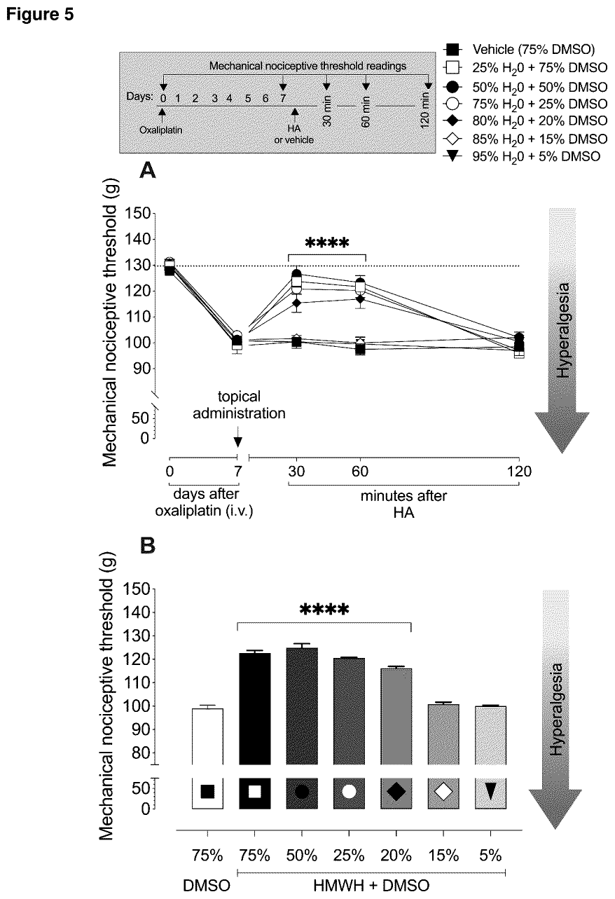

DMSO concentration. We next determined the lower end of the concentration range of DMSO necessary to facilitate transdermal hyaluronan to induce anti-hyperalgesia in rats with CIPN (FIG. 5). Down to a concentration of DMSO of 20%, 500-1200 kDa hyaluronan produced robust anti-hyperalgesia in rats with oxaliplatin CIPN (FIG. 5A). However, when the DMSO concentration was lowered to 15%, 500-1200 kDa hyaluronan no longer produced anti-hyperalgesia. To better visualize the difference in the DMSO concentration we show an area under curve (AUC) 30 and 60 min after topical application (FIG. 5B).

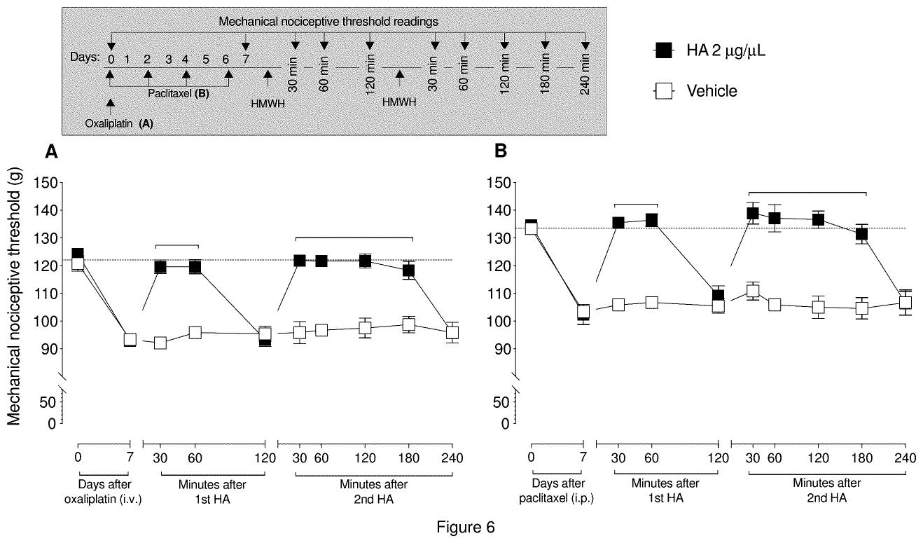

Repeated administration of hyaluronan. Anti-hyperalgesia for oxaliplatin (FIG. 6A) and paclitaxel (FIG. 6B) CIPN, produced by 500-1200 kDa hyaluronan, dissipated by 120 minutes. However, when reapplied at the same site, in male rats with oxaliplatin (FIG. 6A) or paclitaxel (FIG. 6B) CIPN, produced longer lasting anti-hyperalgesia, an effect still present at 180 minutes after the second administration.

Role of molecular weight in hyaluronan anti-hyperalgesia. We have previously shown that while intradermal injection of 500-1200 kDa hyaluronan induced anti-hyperalgesia [12-16; 36], intradermal injection of a low molecular weight hyaluronan (˜1.2 kDa) induced hyperalgesia [13; 14; 34; 36]. Therefore, we next evaluated the range of molecular weights of topical hyaluronan combined with DMSO that produce anti-hyperalgesia. In separate groups of rats with oxaliplatin CIPN, we administered hyaluronan of different molecular weight ranges: 70-120 kDa, 150-300 kDa, 300-500 kDa, 500-1200 kDa, or 1500-1750 kDa. Except for 70-120 kDa hyaluronan, which did not attenuate CIPN hyperalgesia, the other hyaluronan molecular weight ranges tested produced robust anti-hyperalgesia, of similar magnitude and duration (FIG. 7A).

Efficacy of other transdermal delivery enhancers. Since DMSO alone has physiological effects, to demonstrate that hyaluronan requires a transdermal drug delivery enhancer to produce anti-hyperalgesia, male rats received oxaliplatin, and a combination of hyaluronan and protamine (another skin penetration enhancement molecule) administered topically on the dorsum of the hind paw, at the site of nociceptive threshold testing. Topical administration of hyaluronan combined with protamine produced robust anti-hyperalgesia (FIG. 7B). As a further test that transdermal drug delivery enhancers, as a class, facilitate anti-hyperalgesia by topical hyaluronan, a combination of hyaluronan and terpene was administered topically on the dorsum of the hind paw, at the same site. Topical administration of hyaluronan combined with terpene also produced anti-hyperalgesia (FIG. 7C). Importantly, the duration of hyaluronan anti-hyperalgesia when administered with protamine or terpene was much longer than when administered in DMSO vehicle.

Since hyaluronan in protamine vehicle produced longer anti-hyperalgesia, and DMSO showed longer lasting anti-hyperalgesia after the second administration, we determined if hyaluronan combined with protamine, when reapplied at the same site, produces longer lasting anti-hyperalgesia. The second topical administration of hyaluronan combined with protamine produced robust anti-hyperalgesia (FIG. 7D), lasting 5 hours.

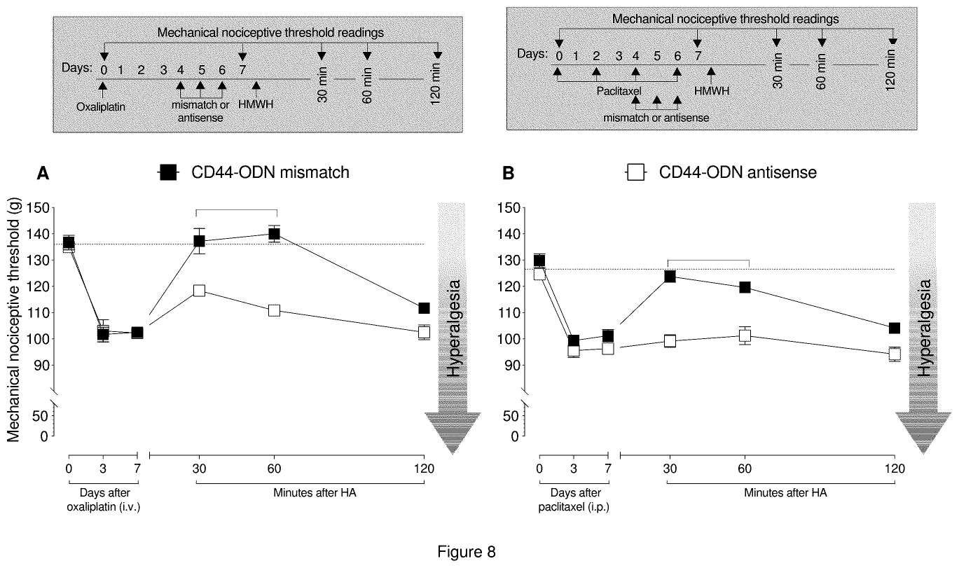

CD44 receptor dependence of anti-hyperalgesia induced by topical hyaluronan. We have previously shown that anti-hyperalgesia induced by intradermal injection of 500-1200 kDa hyaluronan was dependent on its action at nociceptor CD44 [12-14; 34; 36]. Here we determined if the anti-hyperalgesia induced by topical 500-1200 kDa hyaluronan in CIPN is also nociceptor CD44-dependent. Male rats received oxaliplatin (2 mg/kg, i.v.) (FIG. 8A) or paclitaxel (1 mg/kg, i.p., 4 doses every other day) (FIG. 8B). Four days after oxaliplatin or the first injection of paclitaxel, rats received i.t. injections of ODN antisense or mismatch to CD44 mRNA, for 3 consecutive days. On the fourth day, approximately 24 h after administration of the last dose of ODN, topical 500-1200 kDa hyaluronan (2 μg/μL, 30 μL) was applied on the dorsum of the hind paw. In male rats with CIPN, induced by both oxaliplatin and paclitaxel, the anti-hyperalgesia induced by 500-1200 kDa hyaluronan was markedly attenuated in rats treated with ODN antisense to CD44 mRNA (FIGS. 8A and 8B).

### Discussion

A general concern with systemic drug administration is their associated adverse effects, some quite serious [55; 62] that can, in some circumstances, be abrogated by using topical/local routes of administration (Valenta and Almasi-Szabo, 1995). Efficacy of topical drug administration, however, may be limited by poor skin permeation [72; 78]. For this reason, the development of topical drug formulations using transdermal permeation enhancers can be of potential clinical importance [72].

While the topical route of administration may allow some drugs to access dermal tissues, the skin's surface contains a lamellar structure, the stratum corneum, which forms a major barrier to drug penetration [52; 53]. Composed of dead corneocytes embedded in an intercellular lipid matrix consisting of ceramides, free fatty acids, cholesterol, and cholesteryl esters [53; 57], the stratum corneum, is poorly penetrated by large hydrophilic molecules such as high molecular weight hyaluronan.

We have previously demonstrated that the intradermal injection of high molecular weight hyaluronan, which bypasses the stratum corneum barrier to uptake of topically applied drugs, attenuates PGE₂-induced hyperalgesia, in female and male rats [13], and the hyperalgesia in preclinical models of CIPN, induced by paclitaxel and oxaliplatin, in male and gonadectomized, but not gonad intact, female rats [16]. In the present study we observed that when combined with a transdermal permeability enhancer, DMSO, the topical administration of high molecular weight hyaluronan attenuates PGE₂-induced hyperalgesia, demonstrating that the topical application of high molecular weight hyaluronan could be used for the treatment of cutaneous inflammatory pain. To evaluate the potential use of topical high molecular weight hyaluronan in the treatment of neuropathic pain, we first treated rats with oxaliplatin, paclitaxel or bortezomib, representative of three important classes of chemotherapy, which are thought to induce CIPN by different mechanisms [18; 24; 37; 70]. Topical high molecular weight hyaluronan, when co-administered with DMSO, robustly attenuated oxaliplatin, paclitaxel and bortezomib CIPN in male but not in gonad intact female rats. This high molecular weight hyaluronan-induced anti-hyperalgesia, in rats with CIPN, is dependent on its action at CD44, the cognate hyaluronan receptor [9; 34; 65], on nociceptors, as male rats treated intrathecally with ODN antisense against CD44 mRNA, have decreased high molecular weight hyaluronan-induced anti-hyperalgesia [12; 13; 36].

To evaluate the role of sex hormones in the inability of high molecular weight hyaluronan to induce anti-hyperalgesia in female rats with CIPN, we studied ovariectomized female rats and female rats treated intrathecally with ODN antisense against GPR30, a G-protein coupled estrogen receptor found in DRG neurons [29]. In both groups of female rats, topical high molecular weight hyaluronan now robustly attenuated paclitaxel and oxaliplatin CIPN, demonstrating that female sex hormones may act at receptors on nociceptors to suppress the ability of high molecular weight hyaluronan to suppress CIPN, in female rats. Of note in this regard, since many female oncology patients receiving chemotherapy are post-menopausal, with low estrogen levels, topical high molecular weight hyaluronan will be effective in these patients.

When initially administered the anti-hyperalgesic effect of topical high molecular weight hyaluronan lasted approximately 60 minutes. Of note, however, a second application after the effect of the initial application had dissipated, produced longer lasting anti-hyperalgesia. These results demonstrate that repeated application does not produce tolerance to the therapeutic effect of topical hyaluronan.

Since high molecular weight hyaluronan of different weight ranges are used clinically [43], to identify the molecular weight range effective in producing anti-hyperalgesia with topically administered hyaluronan, we used 4 different molecular weight ranges of hyaluronan, in addition to the weight range we previously administered intradermally to produce anti-hyperalgesia [500-1,200 kDa, [12-16; 34; 36]]. Hyaluronan of higher molecular weight (1,500-1,750 kDa) produced a similar anti-hyperalgesic response. However, a somewhat lower molecular weight range of hyaluronan (70-120 kDa) did not induce anti-hyperalgesia, demonstrating that the molecular weight thresholds established by the inventors of the present disclosure provide the art with novel formulations that will be efficacious.

The stratum corneum prevents the transdermal absorption of poorly permeable and/or high molecular weight compounds [30; 80]. Importantly, high molecular weight hyaluronan alone does not penetrate the skin [72]. Here we show that to produce anti-hyperalgesia for inflammatory and neuropathic pain, topical high molecular weight hyaluronan is achieved the addition of a dermal penetration enhancer. It should be noted that DMSO, a transdermal transporter enhancer may, alone, have physiological effects; for example, topically administered (DMSO) has been used to treat systemic inflammation in veterinary medical practice [27; 63; 76]. However, in our study, DMSO alone has no effect on CIPN; nor was high molecular weight hyaluronan dissolved in saline (0.9% of NaCl) able to induce anti-hyperalgesia in rats with CIPN, demonstrating that the combination of high molecular weight hyaluronan, of a restricted molecular weight range, and a transdermal drug delivery enhancer, as disclosed herein, is required for the anti-hyperalgesic effect of topical hyaluronan. To demonstrate the role of the transdermal drug delivery enhancer, we tested different percentages of DMSO in combination with high molecular weight hyaluronan. High molecular weight hyaluronan combined with DMSO in a concentration greater than 20% produced anti-hyperalgesia of similar magnitude. However, concentrations of hyaluronan less than or equal to 15% of DMSO did not show high molecular weight hyaluronan-induced anti-hyperalgesia. Thus, our results demonstrate that a minimum DMSO concentration of 20% is necessary for HMWH to penetrate the stratum corneum and produce its anti-hyperalgesic effect. Finally, demonstrate alternative embodiments wherein transdermal delivery agents other than DMSO are effective, demonstrated that other transdermal drug delivery enhancers would also allow topical high molecular weight hyaluronan to produce hyperalgesia. Herein it is demonstrated that two other chemically dissimilar transdermal drug delivery enhancers, protamine and terpene [21], both also allowed topical high molecular weight hyaluronan to produce anti-hyperalgesia in rats with CIPN. Of note, the duration of anti-hyperalgesia induced by hyaluronan in combination with these other transdermal drug delivery enhancers were dramatically longer than produced by high molecular hyaluronan in DMSO vehicle, and their repeat administration produce further prolongation of their therapeutic effect.

Since the topical administration of high molecular weight hyaluronan, when combined with a transdermal permeability enhancer, induced anti-hyperalgesia in rats with inflammatory and CIPN hyperalgesia, we demonstrate that high molecular weight hyaluronan combined with transdermal drug delivery enhancers provide a potential clinical option for the treatment of acute and chronic inflammatory and neuropathic pain, and could even enhance its efficacy following intra-articular injection in patients with arthritis where its use in current therapy is designed to remain in the joint space, to produce its therapeutic effect by acting as a viscoelastic cushion [25; 31; 40], by allowing hyaluronan to act deeper in the synovium. Intra-articular injection of high molecular weight hyaluronan, combined with a permeability enhancer would allow penetration into the arthritic synovium to act on nociceptors sensitized by inflammatory mediators.

### REFERENCES

[1] Adams J D, Flora K P, Goldspiel B R, Wilson J W, Arbuck S G, Finley R. Taxol: a history of pharmaceutical development and current pharmaceutical concerns. J Natl Cancer Inst Monogr 1993(15):141-147.
[2] Alessandri-Haber N, Yeh J J, Boyd A E, Parada C A, Chen X, Reichling D B, Levine J D. Hypotonicity induces TRPV4-mediated nociception in rat. Neuron 2003; 39(3):497-511.
[3] Altman R D, Moskowitz R. Intraarticular sodium hyaluronate (Hyalgan) in the treatment of patients with osteoarthritis of the knee: a randomized clinical trial. Hyalgan Study Group. J Rheumatol 1998; 25(11):2203-2212.
[4] Alvarez P, Bogen O, Levine J D. Role of nociceptor estrogen receptor GPR30 in a rat model of endometriosis pain. Pain 2014; 155(12):2680-2686.
[5] Alvarez P, Green P G, Levine J D. Role for monocyte chemoattractant protein-1 in the induction of chronic muscle pain in the rat. Pain 2014; 155(6):1161-1167.
[6] Apfel S C, Lipton R B, Arezzo J C, Kessler J A. Nerve growth factor prevents toxic neuropathy in mice. Ann Neurol 1991; 29(1):87-90.
[7] Araldi D, Bogen O, Green P G, Levine J D. Role of Nociceptor Toll-like Receptor 4 (TLR4) in Opioid-Induced Hyperalgesia and Hyperalgesic Priming. J Neurosci 2019; 39(33):6414-6424.
[8] Araldi D, Ferrari L F, Levine J D. Hyperalgesic priming (type II) induced by repeated opioid exposure: maintenance mechanisms. Pain 2017; 158(7):1204-1216.
[9] Aya K L, Stern R. Hyaluronan in wound healing: rediscovering a major player. Wound Repair Regen 2014; 22(5):579-593.
[10] Banach M, Juranek J K, Zygulska A L. Chemotherapy-induced neuropathies—a growing problem for patients and health care providers. Brain Behav 2017; 7(1):e00558.
[11] Bogen O, Alessandri-Haber N, Chu C, Gear R W, Levine J D. Generation of a pain memory in the primary afferent nociceptor triggered by PKCepsilon activation of CPEB. J Neurosci 2012; 32(6):2018-2026.
[12] Bonet I J M, Araldi D, Green P G, Levine J D. Sexually Dimorphic Role of Toll-like Receptor 4 (TLR4) in High Molecular Weight Hyaluronan (HMWH)-induced Anti-hyperalgesia. J Pain 2021.
[13] Bonet I J M, Araldi D, Khomula E V, Bogen O, Green P G, Levine J D. Mechanisms Mediating High-Molecular-Weight Hyaluronan-Induced Antihyperalgesia. J Neurosci 2020; 40(34):6477-6488.
[14] Bonet I J M, Green P G, Levine J D. Sexual dimorphism in the nociceptive effects of hyaluronan. Pain 2020.
[15] Bonet I J M, Khomula E V, Araldi D, Green P G, Levine J D. PI3Kgamma/AKT signaling in high molecular weight hyaluronan (HMWH)-induced anti-hyperalgesia and reversal of nociceptor sensitization. J Neurosci 2021.
[16] Bonet I J M, Staurengo-Ferrari L, Araldi D, Green P G, Levine J D. Second messengers mediating high-molecular-weight hyaluronan-induced antihyperalgesia in rats with chemotherapy-induced peripheral neuropathy. Pain 2021.
[17] Branca J J V, Carrino D, Gulisano M, Ghelardini C, Di Cesare Mannelli L, Pacini A. Oxaliplatin-Induced Neuropathy: Genetic and Epigenetic Profile to Better Understand How to Ameliorate This Side Effect. Front Mol Biosci 2021; 8:643824.
[18] Bruno P M, Liu Y, Park G Y, Murai J, Koch C E, Eisen T J, Pritchard J R, Pommier Y, Lippard S J, Hemann M T. A subset of platinum-containing chemotherapeutic agents kills cells by inducing ribosome biogenesis stress. Nat Med 2017; 23(4):461-471.
[19] Cavaletti G, Bogliun G, Crespi V, Marzorati L, Zincone A, Marzola M, Rota S, Galli A, Tredici P, Tredici G. Neurotoxicity and ototoxicity of cisplatin plus paclitaxel in comparison to cisplatin plus cyclophosphamide in patients with epithelial ovarian cancer. J Clin Oncol 1997; 15(1):199-206.
[20] Cavaletti G, Tredici G, Braga M, Tazzari S. Experimental peripheral neuropathy induced in adult rats by repeated intraperitoneal administration of taxol. Exp Neurol 1995; 133(1):64-72.
[21] Chen J, Jiang Q D, Chai Y P, Zhang H, Peng P, Yang X X. Natural Terpenes as Penetration Enhancers for Transdermal Drug Delivery. Molecules 2016; 21(12).
[22] Cliffer K D, Siuciak J A, Carson S R, Radley H E, Park J S, Lewis D R, Zlotchenko E, Nguyen T, Garcia K, Tonra J R, Stambler N, Cedarbaum J M, Bodine S C, Lindsay R M, DiStefano P S. Physiological characterization of Taxol-induced large-fiber sensory neuropathy in the rat. Ann Neurol 1998; 43(1):46-55.
[23] Cohen M M, Altman R D, Hollstrom R, Hollstrom C, Sun C, Gipson B. Safety and efficacy of intra-articular sodium hyaluronate (Hyalgan) in a randomized, double-blind study for osteoarthritis of the ankle. Foot Ankle Int 2008; 29(7):657-663.
[24] Colvin L A. Chemotherapy-induced peripheral neuropathy: where are we now? Pain 2019; 160 Suppl 1:S1-S10.
[25] Cowman M K, Schmidt T A, Raghavan P, Stecco A. Viscoelastic Properties of Hyaluronan in Physiological Conditions. F1000Res 2015; 4:622.
[26] Cuff C A, Kothapalli D, Azonobi I, Chun S, Zhang Y, Belkin R, Yeh C, Secreto A, Assoian R K, Rader D J, Pure E. The adhesion receptor CD44 promotes atherosclerosis by mediating inflammatory cell recruitment and vascular cell activation. J Clin Invest 2001; 108(7):1031-1040.
[27] Derry S, Conaghan P, Da Silva J A, Wiffen P J, Moore R A. Topical NSAIDs for chronic musculoskeletal pain in adults. Cochrane Database Syst Rev 2016; 4:CD007400.
[28] Dougados M, Nguyen M, Listrat V, Amor B. High molecular weight sodium hyaluronate (hyalectin) in osteoarthritis of the knee: a 1 year placebo-controlled trial. Osteoarthritis Cartilage 1993; 1(2):97-103.
[29] Dun S L, Brailoiu G C, Gao X, Brailoiu E, Arterburn J B, Prossnitz E R, Oprea T I, Dun N J. Expression of estrogen receptor GPR30 in the rat spinal cord and in autonomic and sensory ganglia. J Neurosci Res 2009; 87(7):1610-1619.
[30] El Maghraby G M, Williams A C, Barry B W. Can drug-bearing liposomes penetrate intact skin? J Pharm Pharmacol 2006; 58(4):415-429.
[31] Elmorsy S, Funakoshi T, Sasazawa F, Todoh M, Tadano S, Iwasaki N. Chondroprotective effects of high-molecular-weight cross-linked hyaluronic acid in a rabbit knee osteoarthritis model. Osteoarthritis Cartilage 2014; 22(1):121-127.
[32] Farquhar-Smith P. Chemotherapy-induced neuropathic pain. Curr Opin Support Palliat Care 2011; 5(1):1-7.
[33] Fernandes R, Mazzarello S, Hutton B, Shorr R, Majeed H, Ibrahim M F, Jacobs C, Ong M, Clemons M. Taxane acute pain syndrome (TAPS) in patients receiving taxane-based chemotherapy for breast cancer—a systematic review. Support Care Cancer 2016; 24(8):3633-3650.
[34] Ferrari L F, Araldi D, Bogen O, Levine J D. Extracellular matrix hyaluronan signals via its CD44 receptor in the increased responsiveness to mechanical stimulation. Neuroscience 2016; 324:390-398.
[35] Ferrari L F, Khomula E V, Araldi D, Levine J D. Marked Sexual Dimorphism in the Role of the Ryanodine Receptor in a Model of Pain Chronification in the Rat. Sci Rep 2016; 6:31221.
[36] Ferrari L F, Khomula E V, Araldi D, Levine J D. CD44 Signaling Mediates High Molecular Weight Hyaluronan-Induced Antihyperalgesia. J Neurosci 2018; 38(2):308-321.
[37] Flatters S J L, Bennett G J. Studies of peripheral sensory nerves in paclitaxel-induced painful peripheral neuropathy: evidence for mitochondrial dysfunction. Pain 2006; 122(3):245-257.
[38] Funakoshi T, Yanai A, Shinoda K, Kawano M M, Mizukami Y. G protein-coupled receptor 30 is an estrogen receptor in the plasma membrane. Biochem Biophys Res Commun 2006; 346(3):904-910.
[39] Furuta J, Ariyoshi W, Okinaga T, Takeuchi J, Mitsugi S, Tominaga K, Nishihara T. High molecular weight hyaluronic acid regulates MMP13 expression in chondrocytes via DUSP10/MKP5. J Orthop Res 2017; 35(2):331-339.
[40] Gomis A, Miralles A, Schmidt R F, Belmonte C. Intra-articular injections of hyaluronan solutions of different elastoviscosity reduce nociceptive nerve activity in a model of osteoarthritic knee joint of the guinea pig. Osteoarthritis Cartilage 2009; 17(6):798-804.
[41] Green P G, Dahlqvist S R, Isenberg W M, Miao F J, Levine J D. Role of adrenal medulla in development of sexual dimorphism in inflammation. Eur J Neurosci 2001; 14(9):1436-1444.
[42] Hamers F P, Pette C, Neijt J P, Gispen W H. The ACTH-(4-9) analog, ORG 2766, prevents taxol-induced neuropathy in rats. Eur J Pharmacol 1993; 233(1):177-178.
[43] Haserodt S, Aytekin M, Dweik R A. A comparison of the sensitivity, specificity, and molecular weight accuracy of three different commercially available Hyaluronan ELISA-like assays. Glycobiology 2011; 21(2):175-183.
[44] Huang T L, Chang C C, Lee C H, Chen S C, Lai C H, Tsai C L. Intra-articular injections of sodium hyaluronate (Hyalgan(R)) in osteoarthritis of the knee. a randomized, controlled, double-blind, multicenter trial in the Asian population. BMC Musculoskelet Disord 2011; 12:221.
[45] Kataoka Y, Ariyoshi W, Okinaga T, Kaneuji T, Mitsugi S, Takahashi T, Nishihara T. Mechanisms involved in suppression of ADAMTS4 expression in synoviocytes by high molecular weight hyaluronic acid. Biochem Biophys Res Commun 2013; 432(4):580-585.
[46] Kunitoh H, Saijo N, Furuse K, Noda K, Ogawa M. Neuromuscular toxicities of paclitaxel 210 mg m(−2) by 3-hour infusion. Br J Cancer 1998; 77(10):1686-1688.
[47] McWhinney S R, Goldberg R M, McLeod H L. Platinum neurotoxicity pharmacogenetics. Mol Cancer Ther 2009; 8(1):10-16.
[48] Mestre C, Pelissier T, Fialip J, Wilcox G, Eschalier A. A method to perform direct transcutaneous intrathecal injection in rats. J Pharmacol Toxicol Methods 1994; 32(4):197-200.
[49] Miaskowski C, Paul S M, Mastick J, Abrams G, Topp K, Smoot B, Kober K M, Chesney M, Mazor M, Mausisa G, Schumacher M, Conley Y P, Sabes J H, Cheung S, Wallhagen M, Levine J D. Associations Between Perceived Stress and Chemotherapy-Induced Peripheral Neuropathy and Otoxicity in Adult Cancer Survivors. J Pain Symptom Manage 2018; 56(1):88-97.
[50] Miller K D, Nogueira L, Mariotto A B, Rowland J H, Yabroff K R, Alfano C M, Jemal A, Kramer J L, Siegel R L. Cancer treatment and survivorship statistics, 2019. CA Cancer J Clin 2019; 69(5):363-385.
[51] Mizrahy S, Raz S R, Hasgaard M, Liu H, Soffer-Tsur N, Cohen K, Dvash R, Landsman-Milo D, Bremer M G, Moghimi S M, Peer D. Hyaluronan-coated nanoparticles: the influence of the molecular weight on CD44-hyaluronan interactions and on the immune response. J Control Release 2011; 156(2):231-238.
[52] Mizutani Y, Mitsutake S, Tsuji K, Kihara A, Igarashi Y. Ceramide biosynthesis in keratinocyte and its role in skin function. Biochimie 2009; 91(6):784-790.
[53] Mohd F, Todo H, Yoshimoto M, Yusuf E, Sugibayashi K. Contribution of the Hair Follicular Pathway to Total Skin Permeation of Topically Applied and Exposed Chemicals. Pharmaceutics 2016; 8(4).
[54] Oliveira-Fusaro M C G, Zanoni C I S, Dos Santos G G, Manzo L P, Araldi D, Bonet I J M, Tambeli C H, Dias E V, Parada C A. Antihyperalgesic effect of CB1 receptor activation involves the modulation of P2X3 receptor in the primary afferent neuron. Eur J Pharmacol 2017; 798:113-121
[55] Ottillinger B, Gomor B, Michel B A, Pavelka K, Beck W, Elsasser U. Efficacy and safety of eltenac gel in the treatment of knee osteoarthritis. Osteoarthritis Cartilage 2001; 9(3):273-280.
[56] Pachman D R, Qin R, Seisler D K, Smith E M, Beutler A S, Ta L E, Lafky J M, Wagner-Johnston N D, Ruddy K J, Dakhil S, Staff N P, Grothey A, Loprinzi C L. Clinical Course of Oxaliplatin-Induced Neuropathy: Results From the Randomized Phase III Trial NO8CB (Alliance). J Clin Oncol 2015; 33(30):3416-3422.
[57] Pappas A. Epidermal surface lipids. Dermatoendocrinol 2009; 1(2):72-76.
[58] Parada C A, Yeh J J, Reichling D B, Levine J D. Transient attenuation of protein kinase Cepsilon can terminate a chronic hyperalgesic state in the rat. Neuroscience 2003; 120(1):219-226.
[59] Petrovchich I, Kober K M, Wagner L, Paul S M, Abrams G, Chesney M A, Topp K, Smoot B, Schumacher M, Conley Y P, Hammer M, Levine J D, Miaskowski C. Deleterious Effects of Higher Body Mass Index on Subjective and Objective Measures of Chemotherapy-Induced Peripheral Neuropathy in Cancer Survivors. J Pain Symptom Manage 2019; 58(2):252-263.
[60] Quanhong Z, Ying X, Moxi C, Tao X, Jing W, Xin Z, Li W, Derong C, Xiaoli Z, Wei J. Intrathecal PLC(beta3) oligodeoxynucleotides antisense potentiates acute morphine efficacy and attenuates chronic morphine tolerance. Brain Res 2012; 1472:38-44.
[61] Randall L O, Selitto J J. A method for measurement of analgesic activity on inflamed tissue. Arch Int Pharmacodyn Ther 1957; 111(4):409-419.
[62] Rolf C, Movin T, Engstrom B, Jacobs L D, Beauchard C, Le Liboux A. An open, randomized study of ketoprofen in patients in surgery for Achilles or patellar tendinopathy. J Rheumatol 1997; 24(8):1595-1598.
[63] Roth S H. Letter to the editor: The importance of differentiating between topical NSAIDs. Postgrad Med 2011; 123(5):251-252; author reply 253-254.
[64] Song M J, Wang Y Q, Wu G C. Additive anti-hyperalgesia of electroacupuncture and intrathecal antisense oligodeoxynucleotide to interleukin-1 receptor type I on carrageenan-induced inflammatory pain in rats. Brain Res Bull 2009; 78(6):335-341.
[65] Stern R, Asari A A, Sugahara K N. Hyaluronan fragments: an information-rich system. Eur J Cell Biol 2006; 85(8):699-715.
[66] Su L, Wang C, Yu Y H, Ren Y Y, Xie K L, Wang G L. Role of TRPM8 in dorsal root ganglion in nerve injury-induced chronic pain. BMC Neurosci 2011; 12:120.
[67] Sun J L, Xiao C, Lu B, Zhang J, Yuan X Z, Chen W, Yu L N, Zhang F J, Chen G, Yan M. CX3CL1/CX3CR1 regulates nerve injury-induced pain hypersensitivity through the ERKS signaling pathway. J Neurosci Res 2013; 91(4):545-553.
[68] Taiwo Y O, Coderre T J, Levine J D. The contribution of training to sensitivity in the nociceptive paw-withdrawal test. Brain Res 1989; 487(1):148-151.
[69] Taiwo Y O, Levine J D. Prostaglandin effects after elimination of indirect hyperalgesic mechanisms in the skin of the rat. Brain Res 1989; 492(1-2):397-399.
[70] Tanishima H, Tominaga T, Kimura M, Maeda T, Shirai Y, Horiuchi T. Hyperacute peripheral neuropathy is a predictor of oxaliplatin-induced persistent peripheral neuropathy. Support Care Cancer 2017; 25(5):1383-1389.
[71] Tavianatou A G, Caon I, Franchi M, Piperigkou Z, Galesso D, Karamanos N K. Hyaluronan: molecular size-dependent signaling and biological functions in inflammation and cancer. FEBS J 2019; 286(15):2883-2908.
[72] Tokudome Y, Komi T, Omata A, Sekita M. A new strategy for the passive skin delivery of nanoparticulate, high molecular weight hyaluronic acid prepared by a polyion complex method. Sci Rep 2018; 8(1):2336.
[73] Triantaffilidou K, Venetis G, Bika O. Efficacy of hyaluronic acid injections in patients with osteoarthritis of the temporomandibular joint. A comparative study. J Craniofac Surg 2013; 24(6):2006-2009.
[74] Vigetti D, Karousou E, Viola M, Deleonibus S, De Luca G, Passi A. Hyaluronan: biosynthesis and signaling. Biochim Biophys Acta 2014; 1840(8):2452-2459.
[75] Vigetti D, Viola M, Karousou E, De Luca G, Passi A. Metabolic control of hyaluronan synthases. Matrix Biol 2014; 35:8-13.
[76] Williams A C, Barry B W. Penetration enhancers. Adv Drug Deliv Rev 2004; 56(5):603-618.
[77] Wu P T, Kuo L C, Su F C, Chen S Y, Hsu T I, Li C Y, Tsai K J, Jou I M. High-molecular-weight hyaluronic acid attenuated matrix metalloproteinase-1 and -3 expression via CD44 in tendinopathy. Sci Rep 2017; 7:40840.
[78] Yu Y Q, Yang X, Wu X F, Fan Y B. Enhancing Permeation of Drug Molecules Across the Skin via Delivery in Nanocarriers: Novel Strategies for Effective Transdermal Applications. Front Bioeng Biotechnol 2021; 9:646554.
[79] Zajaczkowska R, Kocot-Kepska M, Leppert W, Wrzosek A, Mika J, Wordliczek J. Mechanisms of Chemotherapy-Induced Peripheral Neuropathy. Int J Mol Sci 2019; 20(6).
[80] Zurdo Schroeder I, Franke P, Schaefer U F, Lehr C M. Development and characterization of film forming polymeric solutions for skin drug delivery. Eur J Pharm Biopharm 2007; 65(1):111-121.

1. A method of treating pain in a subject in need of treatment for a pain condition by the topical administration to the subject of a therapeutically effective amount of hyaluronic acid having a therapeutically effective molecular weight.

2. The method of claim 1, wherein the pain condition is neuropathic inflammatory or stress-induced pain.

3. The method of claim 1, wherein the hyaluronic acid has a molecular weight of at least 120 KDa.

4. The method of claim 1, wherein the hyaluronic acid has a molecular weight of at least 150 KDa.

5. The method of claim 1, wherein topical administration is achieved by application of a composition comprising hyaluronic acid and a transdermal delivery agent.

6. The method of claim 5, wherein the transdermal delivery agent is selected from DMSO, protamine, and terpenes.

7. A method of treating pruritus in a subject in need of treatment therefor by the administration to the subject of a therapeutically effective amount of hyaluronic acid having a therapeutically effective molecular weight.

8. The method of claim 7, wherein the hyaluronic acid has a molecular weight of at least 120 KDa.

9. The method of claim 7, wherein the hyaluronic acid has a molecular weight of at least 150 KDa.

10. The method of claim 7, wherein the hyaluronic acid is applied topically.

11. A composition for the treatment of pain and/or pruritus, comprising
hyaluronic acid having a molecular weight of at least 120 KDa; and
one or more transdermal delivery agents.

12. The composition of claim 11, wherein the one or more transdermal delivery agents comprises DMSO.

13. The composition of claim 12, wherein the composition comprises 20-80% DMSO by volume.

14. The composition of claim 11, wherein the one or more transdermal delivery agents comprises protamine.

15. The composition of claim 11, wherein the one or more transdermal delivery agents comprises one or more terpenes.

16. The composition of claim 15, wherein the one or more terpenes comprises one or more ginkolides.

17. The method of claim 1, wherein the hyaluronic acid is injected into a body cavity.

18. The composition of claim 12, wherein the ratio of hyaluronan to DMSO is less than or equal to about 0.0378 μg hyaluronan per μL DMSO, less than or equal to about 0.035 μg hyaluronan per μL DMSO, less than or equal to about 0.030 μg hyaluronan per μL DMSO, less than or equal to about 0.02677 μg hyaluronan per μL, less than or equal to about 0.025 μg hyaluronan per μL DMSO, less than or equal to about 0.024 μg hyaluronan per μL DMSO, less than or equal to about 0.023 μg hyaluronan per μL DMSO, less than or equal to about 0.022 μg hyaluronan per μL DMSO, less than or equal to about 0.021 μg hyaluronan per μL DMSO, less than or equal to about 0.020 μg hyaluronan per μL DMSO.

19. The method of claim 7, wherein the hyaluronic acid is injected into a body cavity.
